(19) United States
(12) Patent Application Publication
(10) Pub. No.: US 2023/0383016 A1
(43) Pub. Date: Nov. 30, 2023

(54) METHOD OF SELECTING COMPOSITIONS COMPRISING CROSSLINKED HYALURONIC ACID AND A LOW PROPORTION OF SOLUBLE HYALURONIC ACID OF LOW MOLECULAR WEIGHT

(71) Applicant: TEOXANE SA, Geneve (CH)

(72) Inventors: Jimmy FAIVRE, VALSERHONE (FR); François BOURDON, GAILLARD (FR)

(73) Assignee: TEOXANE SA, GENEVE (CH)

(21) Appl. No.: 18/030,947

(22) PCT Filed: Oct. 11, 2021
(86) PCT No.: PCT/EP2021/078065

§ 371 Date: Apr. 7, 2023

(30) Foreign Application Priority Data
Oct. 9, 2020 (EP) 20201186.2

(51) Int. Cl. C08B 37/08

(52) U.S. Cl. CPC C08B 37/0072

(57) ABSTRACT

The invention has for object a method of selecting a composition comprising crosslinked hyaluronic acid (HA) and a low proportion of soluble hyaluronic acid for use in limiting the risk of appearance of an adverse side effect associated with the administration of a composition comprising crosslinked HA.

### FIELD OF THE INVENTION AND BACKGROUND

The present invention relates to the field of compositions, preferably gels, comprising crosslinked hyaluronic acid, notably for soft tissues filling, such as wrinkles filling.

Hyaluronic acid is a linear non-sulfated glycosaminoglycan composed of repeated units of D-glucuronic acid and N-acetyl-D-glucosamine (Tammi R., Agren U M., Tuhkanen A L., Tammi M. Hyaluronan metabolism in skin. Progress in Histochemistry & Cytochemistry 29 (2): 1.-81, 1994). In the human body, its highest occurrence is in the extracellular matrix (ECM) of connective tissues. It is particularly abundant in the dermis of the skin, the synovial fluid of joints and the vitreous body of the eye.

In particular, hyaluronic acid is the major component of the extracellular matrix (ECM). The ECM is a dynamic structure with a structural and regulatory role for the tissues. The ECM gives to the skin its volume, firmness, elasticity and tone. In the skin, hyaluronic acid is primarily synthesized by dermal fibroblasts and epidermal keratinocytes. Through its residues bearing a negative charge, hyaluronic acid acts as a water pump for maintaining the elasticity of the skin. It is noticed that, with age, the amount of hyaluronic acid and its degree of polymerization decreases, resulting in a decrease in the amount of water retained in the connective tissue. Meanwhile, ECM components are degraded, mainly by endopeptidase type enzymes called matrix metalloproteinases or MMPs. Finally, decreases in cellular defenses increase damage and disorders induced by external stresses such oxidative stress.

This aging process leads to the appearance of defects and blemishes of keratinous substances, like wrinkles.

With its excellent physicochemical properties such as biodegradability, biocompatibility, nontoxicity and non-immunogenicity, hyaluronic acid has a wide range of applications and serves as an excellent tool in biomedical field such as rheumatology (e.g. osteoarthritis surgery), ophthalmology (e.g. ocular surgery), cosmetic, aesthetic (e.g. plastic surgery), dermatology, tissue engineering, and drug delivery.

Injectable soft tissue fillers compositions are widely used in the aesthetic field in order to counteract skin depressions and changes due to tissue aging and loss. They help by reducing the intensity of skin folds, wrinkles, lines as well as creating facial volume in specific area. Among all dermal fillers, hyaluronic acid-based gels have garnered increased attention over the last decades due to immediate and natural-looking visual effects on skin as well as being proven to be safe, long-lasting and easy-to-use alone or in combined treatments.

However, hyaluronic acid is known to be highly sensitive to pH variations like strong acidic and alkali pH, high temperatures (e.g. during heat sterilization), oxidation (e.g. reactive oxygen species) and enzymatic activity (Stern R, Kogan G, Jedrzejas M J, et al. The many ways to cleave hyaluronan. Biotechnol Adv 2007; 25:537-57).

As a result, the manufacturing process and notably the crosslinking conditions applied for producing compositions comprising crosslinked hyaluronic acid are prone to drastically affect the native state of hyaluronic acid chains releasing low molecular weight soluble hyaluronic acid.

Although hyaluronic acid is known to be biocompatible, it has been reported in the literature that low molecular weight hyaluronic acid could have potential long-term safety issues in vivo (Cyphert J M, Trempus C S, Garantziotis S. Size Matters: Molecular Weight Specificity of Hyaluronan Effects in Cell Biology. International Journal of Cell Biology 2015; 2015:563818).

Accordingly, compositions comprising crosslinked hyaluronic acid prepared thanks to different manufacturing processes may significantly differ in their final in vivo characteristics with more or less safety issues, in particular inflammatory reactions.

There is always a need to provide compositions comprising crosslinked hyaluronic acid that are safer, i.e. with limited risks of appearance of a side-effect associated with its administration but current in vitro tests are not sufficient for anticipating a safety profile and need to be combined with in vivo tests.

Said in vitro testing includes cytotoxicity tests, cells viability tests and analysis of inflammation markers (gene expression) on dermal cells (fibroblasts) and immune cells (like dendritic cells and macrophages).

Said in vivo testing includes implantation tests in small animals to monitor the appearance of any adverse side effect and histological sections at the implantation site to visualize inflammation markers.

However, such in vivo and in vitro tests are long and expensive and require sacrificing animals for the in vivo ones.

Therefore, in order to avoid or at least minimize these in vivo tests, there is a need to have an in vitro method allowing to better anticipate safety profile as well as mechanical performances of compositions comprising crosslinked hyaluronic acid.

In other words, there is a need for a method for selecting, early in the development process, safe compositions, i.e. compositions with a lower risk of appearance of a side-effect associated with the administration of a composition comprising hyaluronic acid.

### SUMMARY OF THE INVENTION

The present invention fills the abovementioned needs by proposing an in vitro method of selecting a safe composition comprising crosslinked hyaluronic acid, i.e. a composition comprising crosslinked hyaluronic acid with a limited risk of appearance of an adverse side effect, associated with its administration.

As it has been reported that low molecular weight hyaluronic acid could have potential long-term in vivo safety issues, the size of hyaluronic acid chains within a composition can be analysed in order to predict the safety of said composition.

Crosslinked hyaluronic acid comprises hyaluronic acid chains all linked to each others that form an insoluble fraction of long hyaluronic acid chains, on the contrary, hyaluronic acid of low molecular weight is soluble and can be recovered from a composition by extraction for subsequent analysis.

Therefore, in order to predict the behaviour of compositions in situ, analysis of soluble hyaluronic acid can be performed.

It has to be noted that soluble hyaluronic acid includes:
uncrosslinked hyaluronic acid of high molecular weight that can be incorporated within the composition for example to improve its injectability;
small soluble fragments of crosslinked hyaluronic acid formed, notably by fragmentation of crosslinked hyaluronic acid, during the manufacturing process of the composition (due to alkali conditions, heat, sterilization, etc.), particularly due to the crosslinking harsh conditions.

Accordingly, for two compositions prepared with same raw materials, a lower amount of soluble hyaluronic acid of low molecular weight indicates a lower degradation of hyaluronic acid, thus the preservation of integrity of crosslinked hyaluronic acid chains, during the composition preparation process and higher mass-average molecular weights of soluble hyaluronic acid, i.e. longer soluble hyaluronic acid fragments, suggest a lower release of low molecular weight hyaluronic acid which indicate a better conservation of hyaluronic acid chains integrity during the composition preparation process, and at the end a better safety profile.

Moreover, the composition cohesivity relies on the cumulative effect of weak, non-covalent and reversible intermolecular interactions between hyaluronic acid chains, and notably crosslinked hyaluronic acid chains, which dissipates the energy generated by tissue shear or compression. Conserved hyaluronic acid long chains contribute to maximize these interactions.

Thus, a higher mass-average molecular weight of soluble hyaluronic acid suggests a higher cohesivity, and thus a higher capacity to accompany and adapt to muscles movements, such as the ones driving dynamic facial motion.

The inventors have surprisingly found that a composition comprising crosslinked hyaluronic acid and soluble hyaluronic acid, selected as described in claims 1 to 8 is useful for limiting the risk of appearance of an adverse side effect associated with its administration.

In this context, the inventors have developed a method of selecting a composition, preferably a gel, comprising crosslinked hyaluronic acid and a given amount of soluble hyaluronic acid having a molecular weight lower than 50 kDa and/or a given amount of soluble HA having a molecular weight lower than 30 kDa and/or a given the amount of soluble HA having a molecular weight lower than 20 kDa, and optionally a given weight average molecular weight of soluble HA, notably for its use in limiting the risk of appearance of an adverse side effect associated with the administration of such a composition.

Thus, the present invention relates to a method of selecting composition(s) comprising crosslinked hyaluronic acid and soluble hyaluronic acid from a set of compositions comprising crosslinked hyaluronic acid and soluble hyaluronic acid, comprising the steps of:
a) extracting the soluble hyaluronic acid of each composition of the set of compositions by diluting each composition within a solvent usable as mobile phase for Size Exclusion Chromatography (SEC) in order to obtain a diluted composition and filtering each such diluted composition to obtain a filtrate;
b) injecting each filtrate obtained through step a) in SEC column(s) and eluting it through the column(s) to obtain chromatograms;
c) analysing the chromatograms obtained through step b) in order to identify and quantify the soluble hyaluronic acid molecular weights, and notably to determine the amount of the soluble hyaluronic acid having a molecular weight lower than 50 kDa, the amount of the soluble hyaluronic acid having a molecular weight lower than 30 kDa, the amount of the soluble hyaluronic acid having a molecular weight lower than 20 kDa, and optionally the weight average molecular weight of the soluble hyaluronic acid, and,
d) thanks to step c) analysis, selecting composition(s) having:
the lower amount(s) of the soluble hyaluronic acid having a molecular weight lower than 50 kDa in percentage by weight with respect to the total weight of the hyaluronic acid within the composition, or an amount inferior or equal to 5%, preferably inferior or equal to 4%, still preferably inferior or equal to 3%, better still preferably inferior or equal to 2%, by weight with respect to the total weight of the hyaluronic acid within the composition; and/or
the lower amount(s) of the soluble hyaluronic acid having a molecular weight lower than 30 kDa in percentage by weight with respect to the total weight of the hyaluronic acid within the composition, or an amount inferior or equal to 2%, preferably inferior or equal to 1%, by weight with respect to the total weight of the hyaluronic acid within the composition; and/or
the lower amount(s) of the soluble hyaluronic acid having a molecular weight lower than 20 kDa in percentage by weight with respect to the total weight of the hyaluronic acid within the composition, or an amount inferior or equal to 1% with respect to the total weight of the hyaluronic acid within the composition.

Thus, the invention offers a method of selecting composition(s) through in vitro evaluation of their safety profile.

The selection method according to the invention is advantageous compared to known methodologies as it allows to anticipate, at the in vitro stage (i.e. without using animals), quickly and at a low price, potential post-administration adverse side effects. In this context, larger sets of compositions can be studied for selecting a composition of interest which is safe and mechanically efficient. The method according to the invention thus allows a quick selection of safe compositions comprising crosslinked hyaluronic acid, early in the development of a composition, i.e. during in vitro testing and before in vivo evaluation.

A method according to the invention is advantageously a method according to one of the following aspects.

Aspect 1: A method comprising:
a) extracting soluble hyaluronic acid (HA) of each composition of a set of compositions comprising crosslinked HA and soluble HA by:
   diluting each composition within a solvent usable as mobile phase for Size Exclusion Chromatography (SEC) in order to obtain a diluted composition, and filtering each diluted composition in order to obtain a filtrate,
b) injecting each filtrate obtained through step a) in SEC column(s) and eluting it through the column(s) to obtain chromatograms,
c) analysing the chromatograms obtained through step b) in order to identify and quantify soluble HA molecular weights, and
d) selecting the composition(s) comprising crosslinked HA and soluble HA,
   wherein the soluble HA comprises an amount of soluble HA having a molecular weight lower than 50 kDa, and
   wherein the amount of soluble HA having a molecular weight lower than 50 kDa is inferior or equal to 5% compared to the total weight of HA present in the composition,
   wherein the amount of soluble HA having a molecular weight lower than 50 kDa is determined in step c).

Aspect 2: A method comprising:
a) extracting soluble hyaluronic acid (HA) of each composition of a set of compositions comprising crosslinked HA and soluble HA by:
   diluting each composition within a solvent usable as mobile phase for Size Exclusion Chromatography (SEC) in order to obtain a diluted composition, and filtering each diluted composition in order to obtain a filtrate,
b) injecting each filtrate obtained through step a) in SEC column(s) and eluting it through the column(s) to obtain chromatograms,
c) analysing the chromatograms obtained through step b) in order to identify and quantify soluble HA molecular weights, and
d) selecting the composition(s) comprising crosslinked HA and soluble HA,
   wherein the soluble HA comprises an amount of soluble HA having a molecular weight lower than 30 kDa, and
   wherein the amount of soluble HA having a molecular weight lower than 30 kDa is inferior or equal to 2% compared to the total weight of HA present in the composition,
   wherein the amount of soluble HA having a molecular weight lower than 30 kDa is determined in step c).

Aspect 3: A method comprising:
a) extracting soluble hyaluronic acid (HA) of each composition of a set of compositions comprising crosslinked HA and soluble HA by:
   diluting each composition within a solvent usable as mobile phase for Size Exclusion Chromatography (SEC) in order to obtain a diluted composition, and filtering each diluted composition in order to obtain a filtrate,
b) injecting each filtrate obtained through step a) in SEC column(s) and eluting it through the column(s) to obtain chromatograms,
c) analysing the chromatograms obtained through step b) in order to identify and quantify soluble HA molecular weights, and
d) selecting the composition(s) comprising crosslinked HA and soluble HA,
   wherein the soluble HA comprises an amount of soluble HA having a molecular weight lower than 20 kDa, and
   wherein the amount of soluble HA having a molecular weight lower than 20 kDa is inferior or equal to 1% compared to the total weight of HA present in the composition,
   wherein the amount of soluble HA having a molecular weight lower than 20 kDa is determined in step c).

Aspect 4: The method according to aspect 1, wherein the composition(s) selected in step d) is(are) composition(s) comprising crosslinked HA and soluble HA,
wherein the soluble HA comprises an amount of soluble HA having a molecular weight lower than 50 kDa,
wherein the amount of soluble HA having a molecular weight lower than 50 kDa is inferior or equal to 5% compared to the total weight of HA present in the composition, and/or
wherein the soluble HA comprises an amount of soluble HA having a molecular weight lower than 30 kDa,
wherein the amount of soluble HA having a molecular weight lower than 30 kDa is inferior or equal to 2% compared to the total weight of HA present in the composition, and/or
wherein the soluble HA comprises an amount of soluble HA having a molecular weight lower than 20 kDa,
wherein the amount of soluble HA having a molecular weight lower than 20 kDa is inferior or equal to 1% compared to the total weight of HA present in the composition, and
wherein the amount of soluble HA having a molecular weight lower than 50 kDa, the amount of soluble HA having a molecular weight lower than 30 kDa and the amount of soluble HA having a molecular weight lower than 20 kDa are determined in step c).

Aspect 5. The method according to aspect 1, wherein the composition(s) selected in step d) comprise(s) at least 50% by weight of crosslinked HA relative to the total weight of the composition.

Aspect 6. The method according to aspect 1, wherein the soluble HA of the composition(s) selected in step d) has(have) a weight average molecular weight which is higher than 300 kDa, wherein the weight average molecular weight of the soluble HA is determined in step c).

Aspect 7: The method according to aspect 1, wherein the device used to perform the Size exclusion Chromatography (SEC) comprises:
a multiangle light scattering (MALS) detector and a refractive index (RI) detector; and
a liquid chromatography pumping station equipped with a dual set of size exclusion columns adapted for molecules with molecular weights comprised from 500 Da to 20 MDa.

Aspect 8: The method according to aspect 1, wherein in step a) the solvent is an aqueous buffer with a pH ranging between 6 and 8, preferably a sodium nitrate aqueous solution with a pH 7.2 and/or wherein in step b) elution through the column(s) is realized at a flow rate ranging from 0.2 to 0.8 mL/min, preferably from 0.2 to 0.4 mL/min, still preferably of 0.3 mL/min.

Aspect 9: The method according to aspect 1, wherein in step a) each composition is diluted in the solvent at a concentration of 1 mg/mL.

Aspect 10: The method according to aspect 1, wherein, in step a), the diluted compositions are filtered with a filter suitable to separate the soluble HA from insoluble aggregates without having an impact on soluble HA molecular weights, preferably a filter with pores having a diameter of 0.45 μm.

Aspect 11. The method according to aspect 1 comprising an additional step e) of evaluating the mechanical performance of each composition selected in step d) and an additional step f) of selecting composition(s) among the compositions selected in step d) according to its (their) mechanical performances.

Aspect 12. The method according to aspect 11, wherein step e) comprises submitting each composition selected in step d) to oscillatory rheology to determine the elastic modulus G′ and to deliver a G′ integration score and/or to a creep measurement allowing determination of the slope of the deformation curve.

Aspect 13. The method according to aspect 12, wherein, in step f), the composition(s) is(are) selected for having the highest G′ integration score or a G′ integration score higher than or equal to 30.000 Pa².

Aspect 14. The method according to aspect 12, wherein, in step f), the composition(s) is(are) selected for having the highest slope of the deformation curve, preferably having a slope of the deformation curve ≥100.10⁻⁶% sec⁻¹, more preferably ≥150.10⁻⁶% sec⁻¹, still more preferably ≥200.10⁻⁶%/sec⁻¹.

Aspect 15: The method according to aspect 1, wherein the composition(s) selected in step d) consist in a gel and the set of compositions consists in a set of gels.

Aspect 16: The method according to aspect 11, wherein the composition(s) selected in step f) consist in a gel and the set of compositions consists in a set of gels.

Aspect 17: The method according to aspect 11, wherein step d) comprises the following two steps:
d1) selecting the composition(s) comprising crosslinked HA and soluble HA, wherein the soluble HA comprises an amount of soluble HA having a molecular weight lower than 50 kDa, and wherein the amount of soluble HA having a molecular weight lower than 50 kDa is inferior or equal to 5% compared to the total weight of HA present in the composition,
   and optionally wherein the soluble HA comprises an amount of soluble HA having a molecular weight lower than 30 kDa, and wherein the amount of soluble HA having a molecular weight lower than 30 kDa is inferior or equal to 2% compared to the total weight of HA present in the composition,
   and optionally wherein the soluble HA comprises an amount of soluble HA having a molecular weight lower than 20 kDa, and wherein the amount of soluble HA having a molecular weight lower than 20 kDa is inferior or equal to 1% compared to the total weight of HA present in the composition, and
   wherein the amount of soluble HA having a molecular weight lower than 50 kDa, the amount of soluble HA having a molecular weight lower than 30 kDa and the amount of soluble HA having a molecular weight lower than 20 kDa are determined in step c), and
d2) among the composition(s) selected in d1), selecting the composition(s) comprising the soluble HA having the highest weight average molecular weights, and notably 1, 2, 3 or 4 composition(s) having the highest weight average molecular weights or the composition(s) having a weight average molecular weight higher than 300 kDa.

Aspect 18. The method according to aspect 17, wherein, in step a), the set of compositions is a set of gels, each composition is diluted in the solvent at a concentration of 1 mg/mL and the diluted compositions are filtered with a filter suitable to separate the soluble HA from insoluble aggregates without having an impact on soluble HA molecular weights, preferably a filter with pores having a diameter of 0.45 μm, and wherein the composition(s) selected in step f) is(are) a gel.

In one embodiment, the invention relates to (object 1) a method of selecting, from a set of compositions, a composition comprising crosslinked hyaluronic acid (HA) and soluble HA,
wherein the amount of soluble HA having a molecular weight lower than 50 kDa is lower than or equal to 5% by weight of the total weight of HA present in the composition, and/or
wherein the amount of soluble HA having a molecular weight lower than 30 kDa is inferior or equal to 2% by weight of the total weight of HA present in the composition, and/or
wherein the amount of soluble HA having a molecular weight lower than 20 kDa is inferior or equal to 1% by weight of the total weight of HA present in the composition, in particular wherein the amount of soluble HA having a molecular weight lower than lower than or equal to 5% by weight of the total weight of HA present in the composition,
said method comprising the steps of:
a) extracting the soluble HA of each composition of the set of compositions, by diluting each composition within a solvent usable as mobile phase for Size Exclusion Chromatography (SEC) in order to obtain a diluted composition and filtering each such diluted composition to obtain a filtrate,
b) injecting each filtrate obtained through step a) in SEC column(s) and eluting it through the column(s) to obtain chromatograms,
c) analysing the chromatograms obtained through step b) in order to identify and quantify soluble HA molecular weights, and notably to determine the amount of soluble HA having a molecular weight lower than 50 kDa, the amount of soluble HA having a molecular weight lower than 30 kDa, the amount of soluble HA having a molecular weight lower than 20 kDa, and optionally the weight average molecular weight of soluble HA, and,
d) selecting compositions as defined above.

In another embodiment, the invention relates to (object 2) a method according to object 1, wherein the amount of soluble HA having a molecular weight lower than 50 kDa is lower than or equal to 5% by weight of the total weight of HA present in the composition, and
wherein the amount of soluble HA having a molecular weight lower than 30 kDa is lower or equal to 2% by weight compared to the total weight of HA present in the composition and/or the amount of soluble HA having a molecular weight lower than 20 kDa is lower or equal to 1% by weight compared to the total weight of HA present in the composition.

In another embodiment, the invention relates to (object 3) a method according to object 1 or 2, wherein the composition comprises at least 50% by weight of crosslinked HA relative to the total weight of the composition.

In another embodiment, the invention relates to (object 4) a method according to any one of objects 1 to 3, wherein the weight average molecular weight of the soluble HA is higher than 300 kDa.

In another embodiment, the invention relates to (object 5) a method according to any one of objects 1 to 4, wherein the composition is injectable.

In another embodiment, the invention relates to (object 6) a method according to any one of objects 1 to 5, wherein the composition is a gel.

In another embodiment, the invention relates to (object 7) a method according to any one of objects 1 to 6, wherein the composition is sterile, preferentially sterilized by autoclaving.

In another embodiment, the invention relates to (object 8) a method according to any one of objects 1 to 7, wherein the device used to perform the Size exclusion

Chromatography (SEC) Comprises:
a multiangle light scattering (MALS) detector and a refractive index (RI) detector; and
a liquid chromatography pumping station equipped with a dual set of size exclusion columns adapted for molecules with molecular weights comprised from 500 Da to 20 MDa.

In another embodiment, the invention relates to (object 9) a method according to any one of objects 1 to 8, wherein in step a) the solvent is an aqueous buffer with a pH ranging between 6 and 8, preferably a sodium nitrate aqueous solution with a pH 7.2 and/or wherein in step b) elution through the column(s) is realized at a flow rate ranging from 0.2 to 0.8 mL/min, preferably from 0.2 to 0.4 mL/min, still preferably of 0.3 mL/min.

In another embodiment, the invention relates to (object 10) a method according to any one of objects 1 to 9, wherein in step a) each composition is diluted in the solvent at a concentration of 1 mg/mL.

In another embodiment, the invention relates to (object 11) a method according to any one of objects 1 to 10, wherein, in step a), the diluted compositions are filtered with a filter suitable to separate the soluble HA from insoluble aggregates without having an impact on soluble HA molecular weight, preferably a filter with pores having a diameter of 0.45 μm.

In another embodiment, the invention relates to (object 12) a method according to any one of objects 1 to 11, comprising an additional step e) of evaluating the mechanical performance of each composition selected in step d) and an additional step f) of selecting composition(s) among the compositions selected in step d) according to its (their) mechanical performances.

In another embodiment, the invention relates to (object 13) a method according to object 12, wherein step e) comprises submitting each composition selected in step d) to oscillatory rheology to determine the elastic modulus G′ and to deliver a G′ integration score and/or to a creep measurement allowing determination of the slope of the deformation curve.

In another embodiment, the invention relates to (object 14) a method according to object 13, wherein in step f) the composition(s) is(are) selected for having the highest G′ integration score or a G′ integration score higher than or equal to 30.000 Pa².

In another embodiment, the invention relates to (object 15) a method according to object 13, wherein in step f) the composition(s) is(are) selected for having the highest slope of the deformation curve or having a slope of the deformation curve ≥100.10⁻⁶% sec⁻¹, more preferably ≥150.10⁻⁶% sec⁻¹, still more preferably ≥200.10⁻⁶% sec⁻¹.

In another embodiment, the invention relates to (object 16) a method according to any one of objects 1 to 15, wherein the composition consists in a gel and the set of compositions consists in a set of gels.

In another embodiment, the invention relates to (object 17) a method according to any one of objects 12 to 15, wherein step d) comprises the following two steps:
d1) selecting compositions as defined in objects 1 or 2, and
d2) among the compositions selected in d1), selecting the composition(s) comprising the soluble HA having the highest average weight molecular weights, and notably 1, 2, 3 or 4 composition(s) having the highest average weight molecular weights or the composition(s) having an average weight molecular weight higher than 300 kDa.

In another embodiment, the invention relates to (object 18) a method according to object 17, wherein, in step a), the set of compositions is a set of gels, each composition is diluted in the solvent at a concentration of 1 mg/mL and the diluted compositions are filtered with a filter suitable to separate the soluble HA from insoluble aggregates without having an impact on soluble HA molecular weight, preferably a filter with pores having a diameter of 0.45 μm, and
wherein the composition(s) selected in step f) is(are) a gel.

### FIGURES

FIGS. 1A-D: Results of Size Exclusion Chromatography (SEC) analysis of soluble hyaluronic acid released from commercial gels after extraction:
(A) Mass-average molecular weight of released soluble hyaluronic acid,
(B) Percentage of released soluble hyaluronic acid with respect to the total weight of hyaluronic acid within the gels,
(C) Percentage of released soluble hyaluronic acid with respect to the total weight of hyaluronic acid within the gels in the distribution ranges of [0-250 kDa], [0-100 kDa], and [0-30 kDa], and,
(D) ¹H NMR analysis to assess the modification degree of hyaluronic acid within the gels.

FIGS. 2A-C: Conditions and results of the study of the cohesivity of commercial gels under mild shear according to the Gavard-Sundaram cohesivity test:
(A) Schematic of the experiment,
(B) Images of the gels extruded in saline buffer before stirring (t=0 s) and after the end of the experiment (t=30 s), and
(C) Cohesivity scores of the gels according to the 5-grade Cohesivity Scale (a 23 mg/mL solution of non-crosslinked 1.5 MDa hyaluronic acid was used as control).

FIGS. 3A-C: Conditions and results of the study of the mechanical resistance of commercial gels under compression:
(A) Schematics of the experiment,
(B) Compression force profiles of gels (only volumizers gels—RHA 4, VYC-20_{L} and RES_{L} (=RES_{LYFT})—are represented), and
(C) Compression forces values.

FIGS. 4A-D: Results of the rheological characterizations of commercial gels measured at 1 Hz:
(A) Shear elastic modulus, G′, measured at 5 Pa,
(B) Phase angle δ and complex viscosity η*, measured at 5 Pa,
(C) Linear viscoelastic region (LVER), and
(D) Plot of G′ as a function of the applied stress. Only volumizers gels—RHA 4, VYC-20_{L} and RES_{LYFT}—are represented (stresses are represented in a logarithmic scale). The inset is the G′ plot in a linear scale.

FIG. 5: Results of the creep test to assess the Stretch score. Only gels intended for superficial wrinkles filling—RHA 1, VYC-12_{L} and RES_{SV}—are represented.

FIGS. 6A-B: Scores of [A] Strength and [B] Stretch of commercially available gels comprising crosslinked hyaluronic acid.

FIG. 7: Table 5—Rheological properties of all investigated gels.

FIG. 8: Illustration of the principle of a creep measurement.

### DETAILED DESCRIPTION OF THE INVENTION

### Definitions

A “gel” according to the present invention is a network of polymers that is swollen throughout its volume by a fluid. This means that a gel is made up of two phases, one “solid” and the other “liquid”, dispersed in each other. The so-called “solid” phase consists of long polymer molecules connected to each other by weak bonds (for example Hydrogen bonds) or covalent bonds and the “liquid” phase consists of a solvent. When the “liquid” phase used as a solvent is mainly water (for example at least 90%, in particular at least 95%, in particular at least 99% by weight), then the gel is called “hydrogel”. Preferably, the liquid phase includes, in particular, a buffer solution, notably a saline phosphate buffer, allowing advantageously to have a liquid medium with a physiologically acceptable pH, i.e. with a pH between 6.8 and 7.8.

A gel according to the present invention corresponds preferably to a product which has a phase angle δ less than or equal to 45° at 1 Hz for a stress of 5 Pa, advantageously a phase angle δ between 2° and 45°. Advantageously, some gels have a phase angle δ between 20° and 45°.

Preferably, a gel according to the present invention, acceptable for the therapeutic, cosmetic and aesthetic applications covered by the present invention, has a stress at cross-over (or stress at the crossing of modulus G′ and G″) greater than or equal to 50 Pa and an elastic modulus G′ greater than or equal to 20 Pa, preferably from 100 Pa to 2000 Pa, still preferably from 100 Pa to 1000 Pa.

“Sterile” relates to an environment ensuring the safety required for preparing a composition which can be safely injected through the skin or used by topical administration on damaged skin surfaces. It also relates to a composition which is prepared in a sterile environment and/or made sterile with a sterilization method which may be chosen among the ones known by the one skilled in the art. For obvious reasons, it is essential that a composition in accordance with the invention is devoid of any contaminant capable of initiating an undesirable side reaction in the subject.

The term “topical” refers to a composition which is intended to be applied on the skin surface of a subject.

In the expression “effective amount of a composition”, the term “effective amount” relates to the amount of a composition which needs to be administered (e.g. applied on the skin surface to be cared) in order to produce the desired effect (e.g. an anti-aging and/or a caring effect).

The expression “on the skin surface” includes the epidermis of a subject, such as its facial epidermis.

The term “hyaluronic acid” includes hyaluronic acid, its salts such as physiologically acceptable salts such as the sodium salt, the potassium salt, the zinc salt and the silver salt, its derivatives and a mixture thereof. It is also referred as “HA” in its abbreviated form.

The term “mass-average molecular weight” (Mw) of hyaluronic acid is expressed in Daltons (Da) or g/mol. The mass-average molecular weight of a hyaluronic acid can be determined by various methods known by the person skilled in the art, such as by capillary electrophoresis, by size exclusion chromatography (SEC), by high performance gel permeation chromatography (HPGPC) or from a measurement of intrinsic viscosity. In the present text, the expressions “average molecular weight”, “mean molecular weight”, “mean Mw”, “mass-average molecular weight”, “weight-average molecular weight”, “average molar mass” or “mass-average molar mass” have been used interchangeably.

According to the invention, a “low molecular weight” hyaluronic acid is defined as a hyaluronic acid with a molecular weight lower than 50 kDa, preferably lower than 30 kDa, still preferably less than 20 kDa.

A “high mass-average molecular weight” hyaluronic acid refers to a hyaluronic acid with a mass-average molecular weight higher than 300 kDa.

“Water-soluble hyaluronic acid”, “soluble hyaluronic acid”, “extractable hyaluronic acid” and “free hyaluronic acid” are used interchangeably and refer to a hyaluronic acid that can be extracted from a composition comprising a crosslinked hyaluronic acid when the latter is set to swell in an excess of an aqueous or biological medium, such as an aqueous buffer, notably in the conditions as described in the present description.

According to the invention, a “solvent usable as mobile phase for Size Exclusion Chromatography” is an aqueous buffer with a pH ranging between 6 and 8, notably between 6.8 and 7.8, preferably a sodium nitrate aqueous solution with a pH of 7.2 or preferably of 7.0±0.5.

“Water-insoluble hyaluronic acid”, “insoluble hyaluronic acid”, and “non-extractable hyaluronic acid” refer to a hyaluronic acid that cannot be extracted from a composition that includes a crosslinked hyaluronic acid when the composition is set to swell in an excess of an aqueous or biological medium, such as an aqueous buffer, notably in the conditions as described in the present description. It is mainly crosslinked hyaluronic acid.

“Crosslinked hyaluronic acid” means a hyaluronic acid formed by reacting at least one uncrosslinked hyaluronic acid, one of its salts, one of its derivatives or a mixture thereof, with a crosslinking agent under conditions suitable for a crosslinking reaction. Said crosslinked hyaluronic acid may be in form of a powder, a gel, a liquid and/or a solid and preferably is a dense three-dimensional network as obtained just after crosslinking before any swelling step. This expression can refer to one crosslinked hyaluronic acid or a mixture of at least two crosslinked hyaluronic acids as defined in the previous sentence.

The term “crosslinking agent” relates to any compound capable of inducing a linkage, preferably a covalent linkage, between the chains of hyaluronic acid. A crosslinking agent in accordance with the invention is preferably a multifunctional crosslinking agent, more preferably a crosslinking agent with two reactive functions. A crosslinking agent in accordance with the invention may be an epoxy crosslinking agent or a non-epoxy crosslinking agent, preferably an epoxy crosslinking agent.

As a non-epoxy crosslinking agent it can be cited for example: endogenous polyamines, aldehyde, carbodiimide and divinylsulfone.

An epoxy crosslinking agent in accordance with the present invention may preferably be selected from the group consisting of 1,4-butanediol diglycidyl ether (BDDE), 1,2,7,8-diepoxyoctane (DEO), 1,4-bis(2,3-epoxypropoxy)butane, 1,4-bisglycidyloxybutane, 1,2-bis(2,3-epoxypropoxy)ethyl ene, and 1-(2,3-epoxypropyl)-2,3-epoxycyclohexane, and mixtures thereof. Preferably, a crosslinking agent in accordance with the invention is an epoxy crosslinking agent. Still preferably, a crosslinking agent in accordance with the invention is 1,4-butanediol diglycidyl ether (BDDE).

“Uncrosslinked hyaluronic acid” and “non-crosslinked hyaluronic acid” refer to a hyaluronic acid, one of its salts, one of its derivatives or a mixture thereof, that has not been modified with a crosslinking agent and has therefore not undergone a crosslinking reaction.

“Mesotherapy” relates to a procedure comprising multiple injections into the skin of a mixture of one or more ingredients, such as a mixture of crosslinked hyaluronic acid, uncrosslinked hyaluronic acid, minerals and vitamins.

A “physiologically acceptable medium” means a medium devoid of toxicity and compatible with the applications of a composition such as considered in the present invention, and more particularly by topical administration and/or by in vivo injection.

In the context of the present invention, the expression “adverse side effect” refers to a symptom of an immune reaction, in particular a hypersensitivity or an inflammatory reaction, associated with the administration of a composition. An adverse side effect generally occurs at, or close to, the site treated with a composition.

Adverse effects may be of different severity (mild, moderate, or severe), time of appearance (early, intermediate or late, i.e. delayed), nature (ischemic or non-ischemic complications) and prognosis. An “early” adverse side effect occurs from minutes up to one week after treatment with a composition. An “intermediate” adverse side effect occurs from one week to one month after treatment with a composition. A “late” adverse side effect, also said delayed adverse side effect, occurs from one month to years after treatment with a composition.

In particular, in case of the administration of a composition comprising crosslinked hyaluronic acid an adverse side effect may be redness, itching, swelling, pain, indurations, nodules, granulomas, papules, discolorations or a combination thereof. A “Redness” is also called erythema. An “Itching” is also called pruritus. An “Edema” can be transient, persistent or even intermittent, and can be an early, intermediate or delayed event.

An adverse side effect typically resolves within few days. Its etiology is multifactorial, the precise mechanism is not known. Said mechanism seems to depend on the composition applied to the subject, the subject health (allergy, illness, like recent infection e.g. a flu), the drug treatment(s) administered to the subject (e.g. antibiotics, antipyretic, non-steroidal anti-inflammatory drugs, anti-infectious agents), together with the conditions applied by the physician during injection (like depth and area of injection). Concerning the composition applied to the subject, it might for example generate low molecular weight hyaluronic acid with pro-inflammatory properties notably from in situ breakdown of hyaluronic acid of the applied composition. Each of these elements can instigate the chain of events leading to inflammatory reactions by activating immune cells.

“Aesthetic and/or cosmetic uses” and “aesthetic and/or cosmetic applications” are synonymous and can be divided into three broad types: deep, mid and superficial indications, notably deep and superficial indications.

According to the invention, a “deep application” refers to the administration of a composition in the deepest layers of the skin, hypodermis and the deepest part of dermis, and/or below the skin (above the periosteum) for volumizing soft tissues, such as for filling of the deepest wrinkles and/or partially atrophied regions of the face and/or body contour. For such a deep application, among a set of compositions, compositions having highest G′ integration scores need to be selected, in particular more than 10⁵ Pa².

According to the invention, a “superficial application” refers to the administration, for example by mesotherapy, of a composition superficially in the skin, or onto the skin, for treatment of the superficial layers of the skin, epidermis and the most superficial part of dermis, like for reducing superficial wrinkles (also called superficial lines) and/or improving the quality of the skin (such as its radiance, density or structure) and/or rejuvenating the skin. For such a superficial application, among a set of compositions, compositions having the highest slopes of the deformation curve, i.e. the highest Stretch scores, need to be selected. In particular, gels having a Stretch score superior or equal to 100.10⁻⁶ s⁻¹, preferably superior or equal to 150.10⁻⁶ s⁻¹, more preferably superior or equal to 200.10⁻⁶ s⁻¹, will be favoured and selected.

According to the invention, a “mid application” refers to the administration, of a composition into the mid part of the skin for treating mid layers of the skin, like for reducing mid wrinkles. For such mid applications, among a set of compositions, compositions having intermediate properties will be selected, i.e. properties between properties of compositions intended for deep applications and properties of compositions intended for superficial applications. Such compositions are sometimes called “utility fillers” or “mid-plan fillers”.

A “volumizer” is a composition useful in deep applications. It is used for adding volume to a soft tissue, for example for a volumizing effect on the face such as for filling a deep wrinkle on the face.

“Filler”, “filler gel”, and “soft tissues filler gel” are used interchangeably. It can be in particular a “dermal filler gel”, also called “cutaneous filler gel”.

“Dynamic areas of the face” are areas, i.e. zones, which are mobiles on the face, for example the lips zone when the person smiles.

Selection Method According to the Invention

As it has been reported that low molecular weight hyaluronic acid could have potential long-term in vivo safety issues, the size of hyaluronic acid chains within a composition can be analysed in order to predict the safety of said composition.

Crosslinked hyaluronic acid comprises hyaluronic acid chains all linked to each others that form an insoluble fraction of long hyaluronic acid chains, on the contrary, hyaluronic acid of low molecular weight is soluble and can be recovered from a composition by extraction for subsequent analysis. The size of soluble hyaluronic acid can thus be used as a readout of hyaluronic acid chain integrity.

Therefore, in order to predict the behaviour of compositions in situ, analysis of soluble hyaluronic acid can be performed.

Accordingly, for two compositions prepared with same raw materials, a lower amount of soluble hyaluronic acid of low molecular weight indicates a lower degradation of hyaluronic acid, thus the preservation of integrity of crosslinked hyaluronic acid chains, during the composition preparation process and higher mass-average molecular weights of soluble hyaluronic acid, i.e. longer soluble hyaluronic acid fragments, suggests a lower release of low molecular weight hyaluronic acid which indicates a better conservation of hyaluronic acid long chains integrity during the composition preparation process, and at the end a better safety profile.

Moreover, the composition cohesivity relies on the cumulative effect of weak, non-covalent and reversible intermolecular interactions between hyaluronic acid chains, and notably crosslinked hyaluronic acid chains, which dissipates the energy generated by tissue shear or compression. Conserved hyaluronic acid long chains contribute to maximize these interactions.

Thus, a higher mass-average molecular weight of soluble hyaluronic acid suggests a higher cohesivity thus a higher capacity to accompany and adapt to muscles movements, such as the ones driving dynamic facial motion.

The selection method according to the invention is advantageous compared to known methodologies as it allows to anticipate, at the in vitro stage (i.e. without using animals), quickly and at a low price, eventual post-administration adverse side effects. In this context, lager sets of compositions car be studied for selecting a composition of interest which is safe and mechanically efficient. The method according to the invention thus allows a quick selection of safe compositions comprising crosslinked hyaluronic acid, early in the development of a composition, i.e. during in vitro testing and before in vivo evaluation.

In a particular embodiment, the selected composition(s) comprising crosslinked hyaluronic acid and soluble hyaluronic acid is(are) gel(s), preferably hydrogel(s).

In another particular embodiment, the selected composition(s) comprising crosslinked hyaluronic acid and soluble hyaluronic acid is(are) sterile(s), preferentially sterilized by autoclaving.

Analysis Method of the Soluble Hyaluronic Acid Fraction Extracted from Compositions Comprising Crosslinked Hyaluronic Acid

Polydispersity (p/d) and Molecular Weights of Soluble Hyaluronic Acid

Such soluble hyaluronic acid analysis is carry on thanks to Size Exclusion Chromatography (SEC). The analytical concept of this technique is based on separation of polymers chains from a polymer mixture as a function of their size using SEC columns with different porosities. The resulting data inform about the weight average molecular weight (units: Da or g/mol) of the soluble hyaluronic acid extracted from a sample, the soluble hyaluronic acid distribution (polydispersity (p/d)). In addition, the soluble hyaluronic acid fraction is determined for multiple molecular weight limits (20 kDa, 30 kDa, 50 kDa, 100 kDa and 250 kDa) which are of interest since, hyaluronic acids of low molecular weight are associated to the risk of appearance of adverse side effects.

In particular, the analysis of the soluble hyaluronic acid of a composition comprising crosslinked hyaluronic acid is performed using High Performance Liquid Chromatography (HPLC) interfaced with Multiangle Light Scattering (MALS) detector and Refractive Index (RI) Detector (HPLC-SEC-MALS-RI).

For analysing the soluble hyaluronic acid, such soluble fraction has to be extracted from the composition. For that, the content of a composition is diluted into a mobile phase suitable for HPLC-SEC analysis, the diluted composition being then filtered to obtain a filtrate containing the soluble hyaluronic acid.

The soluble hyaluronic acid can be extracted by diluting the composition into a mobile phase suitable for HPLC-SEC analysis, notably at about 25° C. and for about 5 days, and then by centrifugating it, notably at about 4400 rpm for about 10 minutes, and finally by filtering it, notably at about 0.45 mm, to obtain a filtrate.

Preferentially, a neutral aqueous (preferably aqueous buffer) mobile phase with a pH ranging between 6 and 8, notably between 6.8 and 7.8, notably a pH 7.2 or 7.0±0.5. to avoid any additional and artificial hyaluronic acid degradation (due to acidic or alkali conditions) is used as eluent, i.e. as mobile phase suitable for HPLC-SEC analysis. It can be in particular a pH 7.0±0.5 solution of 150 nM sodium nitrate containing 0.02% by weight NaN₃.

Working at a low flow rate ranging between 0.2 to 0.8 mL/min, preferably between 0.2 to 0.4 mL/min, still preferably of 0.3 mL/min, allows the preservation of hyaluronic acid chains in their native state. Indeed, it allows reducing the shearing stress likely damaging hyaluronic acid chains.

In a particular embodiment the method comprises:
a) extracting soluble hyaluronic acid of a composition comprising crosslinked hyaluronic acid and soluble hyaluronic acid by:
   diluting the composition within a solvent usable as a mobile phase for SEC in order to obtain a diluted composition; and
   filtering the diluted composition in order to obtain a filtrate;
b) injecting the filtrate obtained through step a) in SEC column(s) and eluting it through said column(s) to obtain a chromatogram;
c) analysing the chromatogram obtained through step b) in order to identify and quantify soluble hyaluronic acid molecular weights, in particular the amount of soluble hyaluronic acid having a molecular weight lower than 50 kDa and/or the amount of soluble hyaluronic acid having a molecular weight lower than 30 kDa and/or or the amount of soluble hyaluronic acid having a molecular weight lower than 20 kDa.

According to an embodiment of the invention, the step c) analysis comprises in addition the determination of the mass-average molecular weight of the soluble hyaluronic acid.

The device used to perform the SEC according to a particular embodiment of the present method comprises:
✓ a multiangle light scattering (MALS) detector and a refractive index (RI) detector; and
✓ a liquid chromatography, more particularly a high performance liquid chromatography (HPLC), pumping station equipped with a dual set of size exclusion columns adapted for molecules with molecular weights comprised from 500 Da to 20 MDa.

In a particular aspect, the solvent of step a) is an aqueous buffer that have a pH ranging between 6 and 8, preferably a sodium nitrate aqueous solution with a pH 7.2 or a pH 7.0±0.5 and/or the elution of step b) is carried out at a flow rate ranging from 0.2 to 0.8 mL/min, preferably from 0.2 to 0.4 mL/min, still preferably of 0.3 mL/min.

In another embodiment, the composition in step a) is diluted in the solvent at a concentration of 1 mg/mL.

In yet another embodiment, the diluted composition of step a), preferably diluted in the solvent at a concentration of 1 mg/mL, is filtered thanks to a filter suitable to separate the soluble hyaluronic acid from insoluble aggregates without having impact on soluble hyaluronic acid molecular weights, preferably a filter with pores having a diameter of 0.45 μm.

No centrifugation step was required. The lack of centrifugation and the use of 0.45 μm filtration, instead of the usual 0.2 or 0.22 μm filtration which are usually reported, contributed to ensure the mildest extraction of sensitive soluble hyaluronic acid.

Thanks to these particular aspects and embodiments, the method according to the invention preserves the native state of HA (and thus preserves the molecular weight, distribution and proportion of soluble hyaluronic acid having a low molecular weight) and thus allows a proper analysis of the soluble hyaluronic acid contained in the tested compositions.

Modification Degree of Hyaluronic Acid

The “Modification degree” (MoD) is the molar amount of the crosslinking agent bound to hyaluronic acid by one or more of its extremities, expressed by 100 moles of hyaluronic acid repeating units within the composition. It can be determined by methods known by the skilled person such as Nuclear Magnetic Resonance (NMR) spectroscopy. For example, a 1% MoD means that there is one crosslinking agent molecule for 100 repeating units of hyaluronic acid within the composition.

The gold standard technique to measure MoD of compositions, in particular gels, comprising hyaluronic acid crosslinked by a crosslinker is Proton Nuclear Magnetic Resonance (¹H NMR) analysis.

Basically, the composition is dried (by precipitation and/or freeze-drying) to remove water and replace it with deuterate water (suitable solvent for NMR of hydrophilic compounds). For practical reasons, the compositions are degraded (enzymatically or in presence of HCl or NaOH) to obtain a low-viscosity solution suitable for NMR analysis. The resulting spectrum gives information about the ratio of the amount of crosslinker with respect to the amount of HA that leads, after meticulous analysis, to the MoD.

Methods for Evaluating the Mechanical Performances of a Composition Comprising Crosslinked Hyaluronic Acid or a Salt Thereof

As the primary function of all compositions comprising crosslinked hyaluronic acid intended to be used in the aesthetic field is to fill skin wrinkles and restore facial volumes with a good bio-integration, their mechanical behavior is a key feature of their clinical use and performance. It is therefore essential to characterize their rheological profiles accurately.

An object of the invention is a method of selecting composition(s) comprising crosslinked hyaluronic acid as described above, wherein a mechanical evaluation process is implemented on a set of said compositions for selecting composition(s) according to its (their) mechanical performances, in particular as a function of its (their) G′ integration score and/or as a function of its (their) slope of deformation curve obtained through a creep measurement.

In particular such a method is implemented for selecting soft tissues filler gel(s), comprising crosslinked hyaluronic acid.

Advantageously, the creep measurement is used in order to differentiate compositions for which G′ integration scores are similar.

The linear viscoelasticity properties of compositions comprising crosslinked hyaluronic acid, in particular of soft tissues filler gels, may be characterized in oscillatory rheology with a deformation (strain) or stress sweep, via in particular the measurement of their elastic modulus G′ (in Pa), of their viscous modulus G″ (in Pa, also called loss modulus) and of their phase angle δ (in °, tan δ=G″/G′).

The elastic modulus G′, also known as the “storage modulus”, measures the energy returned by the composition/gel when it is subjected to weak reversible deformations, for example during an oscillatory stress sweep test under rheometer at a frequency of 1 Hz and a stress of 5 Pa.

The phase angle δ characterizes the degree of viscoelasticity of a material: it varies between 0° for a 100% elastic material (under a stress, all the deformation energy is returned by the material, that is to say it regains its initial shape) and 90° for a 100% viscous material (under a stress, all the deformation energy is lost by the material, that is to say that it flows and completely loses its initial shape).

A dermal filler gel must be predominantly elastic in order to ensure filling properties, that is to say that its δ must be preferably less than 45°.

The rheological parameters of the compositions/gels that are customarily most used are the elastic modulus G′ and viscous modulus G″ and the phase angle S. These data are obtained in oscillatory rheology and are normally given in the linear viscoelasticity zone, where G′, G″ and δ are relatively constant; such a measurement does not reflect all of the mechanical stresses and deformations to which a filler gel is subjected depending on its function.

WO2016150974, the content of which is incorporated by reference, introduces two rheological parameters to evaluate the macroscopic characteristics of fillers, namely the “the G′ integration score” and the “Creep measurement”.

The “G′ integration score” or “Strength score” and the “Creep measurement” or “Stretch score” are useful tools for characterizing and selecting gels in the laboratory, making it possible to limit in vivo tests at the selection stage during the development of a product.

The measurement of the Strength and the Stretch scores facilitate the development of compositions comprising crosslinked hyaluronic acid, such as filler gels and in particular dermal filler gels, and in particular makes possible to easily differentiate several gels in order to retain the one or those having the most advantageous properties with respect to the desired result.

Evaluating the mechanical performance of a composition comprising crosslinked hyaluronic acid, such as filler gels and in particular dermal filler gels, comprises the step consisting in subjecting a sample of said composition to oscillating mechanical stresses making it possible to determine the elastic modulus G′ and to deliver a score representative of the integration of G′ over the stress and/or the strain within a stress and/or the strain interval that includes values of the modulus G′ encountered within the linear viscoelasticity plateau and beyond.

G′ Integration Score or Strength Score

The G′ integration score makes it possible to characterize the mechanical performance of the composition/gel, since the result takes into account not only the level of elastic modulus G′, but also the width of the plateau, that is to say the width of the strain or stress range for which the gel is capable of conserving a high modulus G′. This approach thus makes it possible to describe as “resilient” a gel capable of withstanding a wider range of strain or stress. It is possible to integrate the modulus G′ from a low deformation or stress, for example 1 Pa, which corresponds to the lower limit, up to the upper limit which may be set in various ways.

Thus, the G′ integration score is derived from the integration of G′ over the stress and/or the deformation within a stress and/or deformation interval (integration interval). The integration interval needs to be wide enough to include values of the modulus G′ encountered beyond the linear viscoelasticity plateau.

The integration interval may have, as upper limit, the deformation and/or stress values taken at a point where the modulus G′ has decreased relative to its value in linear regime, in particular any point in the range of decrease of G′ between the end of the plateau and the crossover. The upper limit corresponds preferably to a decrease of at least 10% of the modulus G′ relative to its average value over the plateau (linear viscoelasticity range, LVER). The upper limit may advantageously be taken at the crossover point.

The point referred to as the “cross-over point” is that where the curves giving G′ and G″ cross. The stress and strain at this point are those starting from which a material, predominantly elastic at the lower stresses and strains, enters the flow region.

The lower limit of the integration interval is preferably taken, for the lowest stress and/or deformation values of the measurement, within the linear viscoelasticity range of the modulus G′.

Knowledge of the G′ integration score based on the integration of the modulus G′ proves to be invaluable for comparing several compositions/gels and thus facilitating the selection thereof as a function of the applications.

Accordingly, a method of selecting a composition comprising a crosslinked hyaluronic acid according to the invention preferably comprises a complementary analysis in oscillatory rheology and in a preferred aspect of the present invention, when comparing several compositions/gels to select one intended to volumizing applications, the compositions/gels having higher G′ integration score will be favoured and selected.

For example, for a deep application (e.g. application of filling deep wrinkles or atrophied regions of the face), it is advisable to choose compositions/gels having a high integration score, whereas those having a lower score could be enough for a superficial application (e.g. filling moderate or fine facial wrinkles).

As an example, compositions having a G′ integration score ≥30.000 Pa² will be favoured and selected for volumizing applications.

The integration may be a single integration and may be carried out over the stress, or as a variant over the deformation. The integration may also be a double integration and may be carried out over the stress and the deformation strain.

This parameter is of paramount importance for materials (compositions/gels) subjected to dynamic zones since it exhibits the range of stress or deformation the composition/gel can effectively withstand its viscoelastic properties (notably its G′).

Creep Measurement or Stretch Score

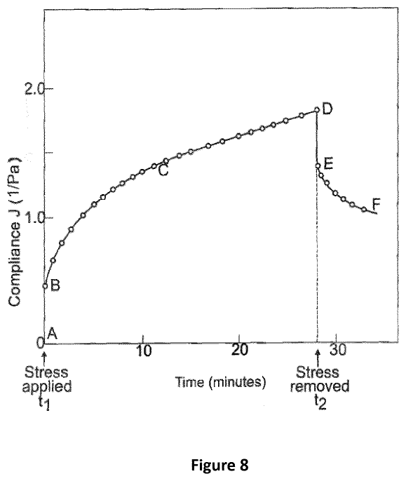

A constant and continuous stress is applied to the gel (unlike the measurement of G′ and G″ where the stress is oscillating) over a given time, and the reaction (deformation) of the composition/gel is measured. The typical type of curve obtained is such as represented in FIG. 8.

The given stress is applied between t1 and t2, and the deformation of the gel is measured over time. It is also possible to measure the elastic compliance J in Pa⁻¹, which gives the same type of curve. After an instantaneous and delayed elastic deformation region, a straight line is observed, the slope of which may be measured. The slope is even greater when the composition/gel creeps easily. In order to measure the creep, it is possible to use the same equipment as the one for measuring the moduli G′ and G″, but operating said equipment (e.g. rheometer) in “creep” (and not oscillating) mode.

For the measurements, a constant stress, for example of 5 Pa, is applied over at least 300 s, preferably 450 s.

The slope of the deformation curve is expressed preferably in s⁻¹, as the ratio between imposed stress G (Pa) and viscosity η (Pa·s): σ/η.

A hyaluronic acid gel with a high resilience, i.e. having a high G′ integration score, may be expected to also be less malleable, i.e. less disposed to creep. This is true in a general manner, that is to say that in a same range of products, or for various hyaluronic acid gels having very different indications, the products having a high G′ integration score generally have a slope of the deformation curve that is lower than that of products having a lower G′ integration score.

The measurement of the creep makes it possible to differentiate products for which the G′ integration scores are similar.

Thus, two products having substantially the same “mechanical resilience” or “Strength score” (G′ integration score), will generally have filling performances and resistance to degradation performances which are quite similar. However, a product that has a significantly higher slope of the creep will have the advantage of being more easily injectable or malleable thus improving the comfort of the patient and giving a more natural effect by adapting to the dynamic areas of the face.

This is an impression which is confirmed by the practitioners during the use of such products. The patients also describe a more natural effect, and the reduction, or even the absence, of discomfort after the injection session.

The combination of the calculation of the integration score and of the slope of the deformation curve therefore gives information, in an overall and thorough manner, on the behaviour of the composition/gel, both on its behaviour “in place” or in situ (durability, strength of the composition/gel, firmness and mechanical resilience), and on its behaviour in a shaping situation (placement of the product during injection, deformation forced by the dynamics areas of the face, natural effect).

It is thus possible to select “2-in-1” products that bring together two a priori antimonic features, namely both a high resilience (the compositions/gels are capable of maintaining their structure and their function despite the stresses undergone) and a good malleability for an optimal and natural shaping of the composition/gel, and for following the dynamics areas of the face. This selection may be made before any in vivo tests.

Accordingly, preferably, a method of selecting a composition comprising a crosslinked hyaluronic acid according to the invention comprises a complementary creep measurement.

According to the present invention, compositions/gels having higher slope of the deformation curve will be favoured and selected, especially for superficial applications, notably in dynamic areas of the face, such as reducing superficial lines or improving the quality of the skin (such as its radiance, density or structure).

In particular the selection of compositions having a Stretch score ≥100.10⁻⁶ s⁻¹, preferably ≥150.10⁻⁶ s⁻¹, more preferably ≥200.10⁻⁶ s⁻¹ will be favoured and selected for superficial applications (e.g. for reducing superficial lines or improving the quality of the skin), notably in dynamic areas of the face.

Global Mechanical Evaluation

Taking into account both the G′ integration score and the Stretch score gives information on the behaviour of the composition/gel not only in situ, especially in terms of durability, firmness and mechanical power, but also on its behaviour in the positioning situation, especially in terms of placement of the composition/gel during the injection, and of deformation forced by the dynamic areas of the face.

Information representative of the G′ integration score may be printed or displayed on an information medium, in particular a notice, a packaging of the composition/gel, an information or advertising panel, a commercial or medical brochure, a television screen or mobile telephone screen. This may give information on the performances of the composition/gel. Where appropriate, information representative of the slope of the deformation curve may also be printed or displayed, for example alongside the information representative of the G′ integration score.

Measurement of the creep makes it possible to evaluate the behaviour of the composition/gel in a non-linear regime, where it is subjected to a continuous stress in the same direction. In other words, it is a deformation forced by the application of a continuous stress which causes the composition/gel to creep.

Measurement of the creep gives information on the ability of the composition/gel to deform under a stress. Concretely, a deformation curve of the tested composition/gel over time is produced. It is important for a composition/gel to be able to be injected easily through a fine needle, to be positioned correctly in the injection site and according to the stresses applied by the practitioner, and to adapt to the stresses and to the dynamic areas of the face, so as to give the effect of natural filling.

The situation is summarized in the Table below:

| Types of | Oscillatory rheology | |
|---|---|---|
| measurement | measurement | Creep measurement |
| Quantities | G’, crossover, δ | Immediate/delayed elastic |
| usually | (small deformations, | deformations, viscous |
| associated | linear | deformations (broad and |
| | viscoelasticity) | irreversible deformations, |
| | | non-linear |
| | | viscoelasticity) |
| Use within the | The integration of the | The greater the slope of the |
| context of the | modulus G’ makes it | deformation curve, the |
| invention | possible to obtain an | more easily deformable/ |
| | indication of mechanical | malleable the |
| | resilience, i.e. the ability | composition/gel is, in the |
| | of the composition/gel | situation of shaping via an |
| | to maintain its structure | imposed stress (e.g. |
| | over a broad stress | modelling of the practitioner, |
| | range | facial movements to which the |
| | | composition/gel must adapt) |

Method of Selecting a Composition Comprising a Crosslinked Hyaluronic Acid

The present invention provides a method of selecting composition(s) comprising crosslinked hyaluronic acid (HA) and soluble HA from a set of compositions comprising crosslinked HA and soluble HA comprises the steps of:
a) extracting the soluble HA of each composition of the set of compositions by diluting each composition within a solvent usable as mobile phase for Size Exclusion Chromatography (SEC) in order to obtain a diluted composition and filtering each such diluted composition to obtain a filtrate;
b) injecting each filtrate obtained through step a) in SEC column(s) and eluting it through the column(s) to obtain chromatograms;
c) analysing the chromatograms obtained through step b) in order to identify and quantify soluble HA molecular weights, and notably to determine the amount of soluble HA having a molecular weight lower than 50 kDa, the amount of soluble HA having a molecular weight lower than 30 kDa, the amount of soluble HA having a molecular weight lower than 20 kDa, and optionally the weight average molecular weight of soluble HA, and,
d) thanks to step c) analysis, selecting composition(s) having:
the lower amount(s) of soluble HA having a molecular weight lower than 50 kDa in percentage by weight with respect to the total weight of HA within the composition preferably an amount inferior or equal to 5%, preferably inferior or equal to 4%, still preferably inferior or equal to 3%, better still preferably inferior or equal to 2% by weight with respect to the total weight of HA within the composition; and/or
the lower amount(s) of soluble HA having a molecular weight lower than 30 kDa in percentage by weight with respect to the total weight of HA within the composition preferably an amount inferior or equal to 2%, preferably to inferior or equal 1% by weight with respect to the total weight of HA within the composition; and/or
the lower amount(s) of soluble HA having a molecular weight lower than 20 kDa in percentage by weight with respect to the total weight of HA within the composition preferably an amount inferior or equal to 1% by weight with respect to the total weight of HA within the composition.

Thus, the invention offers a method of selecting composition(s) through in vitro evaluation of their safety profile.

The present invention relates in particular to a method of selection of a composition comprising crosslinked hyaluronic acid that can be used in a cosmetic or aesthetic application, for example for volumizing soft tissues or for treating the superficial layers of the skin.

The composition(s) to be selected is(are) preferably a gel(s), preferably hydrogel(s) like soft tissues filler gel(s) and, in particular dermal filler gel(s), which is(are) preferably injectable.

Preferably, the composition(s) to be selected is(are) injectable(s).

The composition(s) to be selected is(are) preferably sterile, still preferably sterilized by autoclaving.

Preferably, the method of selection according to the invention comprises in step d) selecting composition(s) with an amount of soluble hyaluronic acid having molecular weight lower than 50 kDa lower than or equal to 5%, preferably lower than or equal to 4%, still preferably lower than or equal to 3%, better still preferably lower than or equal to 2% by weight with respect to the total weight of hyaluronic acid within the composition.

The method of selection according to the invention advantageously comprises, in step d), selecting composition(s) with soluble hyaluronic acid having the highest mass-average molecular weight(s), preferably having a mass-average molecular weight higher than 300 kDa. The mass-average molecular weight of the soluble hyaluronic acid is determined in step c).

More preferably, the method of selection according to the invention comprises in step d) selecting composition(s) with an amount of soluble hyaluronic acid having molecular weight lower than 50 kDa lower than or equal to 5%, preferably lower than or equal to 4%, still preferably lower than or equal to 3%, better still preferably lower than or equal to 2% by weight with respect to the total weight of hyaluronic acid within the composition and with a soluble hyaluronic acid having a mass-average molecular weight which is higher than 300 kDa.

The method of selection according to the invention advantageously comprises, in step d), selecting composition(s) comprising at least 50% by weight of insoluble hyaluronic acid with respect to the total weight of hyaluronic acid within the composition.

Preferably, said step d) comprises the following two steps:
d1) selecting the composition(s) comprising crosslinked hyaluronic acid and soluble hyaluronic acid,
wherein the soluble hyaluronic acid comprises an amount of soluble hyaluronic acid having a molecular weight lower than 50 kDa lower than or equal to 5%, preferably lower than or equal to 4%, still preferably lower than or equal to 3%, better still preferably lower than or equal to 2% by weight with respect to the total weight of hyaluronic acid present in the composition,
and optionally wherein the soluble hyaluronic acid comprises an amount of soluble hyaluronic acid having a molecular weight lower than 30 kDa lower or equal to 2%, preferably lower than or equal to 1% by weight with respect to the total weight of hyaluronic acid within the composition,
and optionally wherein the soluble hyaluronic acid comprises an amount of soluble hyaluronic acid having a molecular weight lower than 20 kDa lower than or equal to 1% by weight with respect to the total weight of hyaluronic acid within the composition, and
wherein the amount of soluble hyaluronic acid having a molecular weight lower than 50 kDa, optionally lower than 30 kDa and optionally lower than 20 kDa is(are) determined in step c), and
d2) among composition(s) selected in d1), selecting
   composition(s) comprising the soluble hyaluronic acids having the highest mass-average molecular weights, notably selecting the 1, 2, 3 or 4 composition(s) having the highest mass-average molecular weights, or
   composition(s) having an average weight molecular weight higher than 300 kDa.

Preferably, the method of selection according to the invention comprises an analysis of polydispersity (p/d) and molecular weights of soluble hyaluronic acids of the set of compositions. Such analysis is(are) preferably executed as described in the above section “analysis method of the soluble hyaluronic acid fraction extracted from compositions comprising crosslinked hyaluronic acid”.

Where applicable, selecting step d) comprises selecting composition(s) having:
the lower amount(s) of soluble HA having a molecular weight lower than 50 kDa in percentage by weight with respect to the total weight of HA within the composition preferably an amount inferior or equal to 5%, preferably inferior or equal to 4%, still preferably inferior or equal to 3%, better still preferably inferior or equal to 2% by weight with respect to the total weight of HA within the composition; and/or,
the lower amount(s) of soluble HA having a molecular weight lower than 30 kDa in percentage by weight with respect to the total weight of HA within the composition preferably an amount inferior or equal to 2%, preferably inferior or equal to 1% by weight with respect to the total weight of HA within the composition; and/or,
the lower amount(s) of soluble HA having a molecular weight lower than 20 kDa in percentage by weight with respect to the total weight of HA within the composition preferably an amount inferior or equal to 1% by weight with respect to the total weight of HA within the composition,
before or after, preferably before, selecting composition(s) with soluble hyaluronic acid having the highest mass-average molecular weight(s), preferably having a mass-average molecular weight higher than 300 kDa.

Preferably, the method of selecting according to the invention comprises an additional step e) of evaluating the mechanical performance of each composition selected in step d) and an additional step f) of selecting composition(s) among compositions selected in step d) according to its (their) mechanical performances. Preferably, the composition(s) selecting in step f) is(are) gel(s).

Selecting step f) may be carried out before or after selecting step d), preferably after.

Preferably, said step e) comprises submitting composition(s) to
oscillatory rheology to determine their elastic modulus G′ and to deliver a G′ integration score and/or to
a creep measurement to determine the slope of their deformation curve.

Preferably, said step f) comprises the selection of composition(s) having:
the highest G′ integration score; or,
a G′ integration score higher than or equal to 30.000 Pa².

Also preferably, said step f) comprises the selection of composition(s) having:
the highest slope of deformation curve; or,
a slope of deformation curve higher than or equal to 100.10⁻⁶ sec⁻¹, more preferably higher than or equal to 150.10⁻⁶ sec⁻¹, still more preferably higher than or equal to 200.10⁻⁶ sec⁻¹.

When there is an additional step e) of evaluating the mechanical performance of each composition of the set of compositions, selecting step d) may be implemented before or after selecting step f), preferably before.

Preferably, where applicable, the method of selecting comprises the step of selecting d) then f) and step d) comprises first the selection of composition(s) according to its (their) amount of soluble HA having a molecular weight lower than 50 kDa and/or a molecular weight lower than 30 kDa and/or a molecular weight lower than 20 kDa and second the selection of composition(s) according to its (their) mass-average molecular weight of soluble HA.

Generally, compositions/gels with high HA concentrations are injected in lower quantities than compositions/gels with lower HA concentrations. In this way, for two compositions selected according to the invention, one having a higher total HA concentration than the other and both having a same percentage of soluble HA with respect to the total weight of HA within the composition, quantities of soluble low molecular weight HA materially injected comply both with the objective of limiting the risk of appearance of adverse side effect associated with the administration of a HA-based composition.

Uses

It is also described the use of composition(s) selected by the method according to the invention for the manufacture of a cosmetic, aesthetic or medicinal product intended to be used for limiting the risk of appearance of an adverse side effect associated with its administration.

It is also described composition(s) selected by the method according to the invention for use in limiting the risk of appearance of an adverse side effect associated with its administration.

It is also described a method for limiting adverse side effect potency of a composition comprising crosslinked hyaluronic acid, said method comprising selecting composition(s) by the selecting method according to the invention.

It is also described a method for limiting the risk of appearance of an adverse side effect associated with the administration of a composition comprising crosslinked hyaluronic acid, said method comprising selecting composition(s) by the selecting method according to the invention.

The composition(s) selected by the method according to the invention is more particularly intended to be used in a cosmetic or aesthetic application, such as volumizing soft tissues, for example for filling of the deepest wrinkles and/or partially atrophied regions of the face and/or body contour; or treating the superficial layers of the skin, for example for reducing superficial wrinkles and/or rejuvenating the skin and/or improving the skin quality.

### EXAMPLES

Material

According to the manufacturer and the technology employed, products are prepared under different conditions.

Table 1 lists the investigated commercially available compositions comprising crosslinked hyaluronic acid according to their names, manufacturer, manufacturing technology, initial hyaluronic acid concentrations within the syringes and indications.

TABLE 1

| | | | HA | |
|---|---|---|---|---|
| | | | concen- | |
| | | | tration | |
| | Manu- | | (mg/ | |
| Product | facturer | Technology | mL) | Indication |
| Teosyal RHA 1 | Teoxane SA | Preserved | 15 | Filling |
| (RHA1) | | Network | | superficial |
| | | | | wrinkles |
| Teosyal RHA 2 | Teoxane SA | Preserved | 23 | Filling mid to- |
| (RHA2) | | Network | | deep wrinkles |
| Teosyal RHA 3 | Teoxane SA | Preserved | 23 | Filling mid to- |
| (RHA3) | | Network | | deep wrinkles |
| Teosyal RHA 4 | Teoxane SA | Preserved | 23 | Volumizing |
| (RHA4) | | Network | | |
| Juvéderm Volite | Allergan | Vycross | 12 | Filling |
| (VYC-12L) | | | | superficial |
| | | | | wrinkles |
| Juvéderm | Allergan | Vycross | 15 | Filling |
| Volbella | | | | superficial |
| (VYC-15L) | | | | wrinkles |
| Juvéderm Volift | Allergan | Vycross | 17.5 | Filling mid to- |
| (VYC-17.5L) | | | | deep wrinkles |
| Juvéderm | Allergan | Vycross | 20 | Volumizing |
| Voluma | | | | |
| XC | | | | |
| (VYC-20L) | | | | |
| Juvéderm | Allergan | Hylacross | 24 | Filling mid to- |
| Ultra XC | | | | deep wrinkles |
| Juvéderm | Allergan | Hylacross | 24 | Filling mid to- |
| Ultra Plus XC | | | | deep wrinkles |
| Restylane | Galderma | NASHA | 20 | Filling |
| Skinboosters | | | | superficial |
| Vital | | | | wrinkles |
| (RES_{SV}) | | | | |
| Restylane | Galderma | NASHA | 20 | Filling mid |
| Lidocaine | | | | to-deep |
| (RES) | | | | wrinkles |
| Restylane Lyft | Galderma | NASHA | 20 | Volumizing |
| (RES_{LYFT}) | | | | |
| Restylane | Galderma | OBT/XPresHAn | 20 | Filling mid to- |
| Refyne | | | | deep wrinkles |
| (RES_{REF}) | | | | |
| Restylane | Galderma | OBT/XPresHAn | 20 | Filling mid to- |
| Defyne | | | | deep wrinkles |
| (RES_{DEF}) | | | | |

Methods

Analysis of the Soluble Hyaluronic Acid Fraction of Gels

Molecular Weights

In this experiment, the analysis of the soluble hyaluronic acid of gels comprising crosslinked hyaluronic acid is performed using High Performance Liquid Chromatography interfaced with Multiangle Light Scattering detector and Refractive Index Detector (HPLC-SEC-MALS-RI, ASTRA software (Wyatt Technology Corp.).

The samples are diluted at 1 mg/mL of hyaluronic acid according to their initial hyaluronic acid concentration into the SEC mobile phase, a filtered 150 mM Sodium Nitrate solution (pH 7.2). Said diluted mixture consists of insoluble and large crosslinked hyaluronic acid and soluble hyaluronic acid. The soluble hyaluronic acid portion of the samples was left to release over 5 days under orbital stirring to avoid any artificial soluble hyaluronic acid production. The soluble portion is separated from the insoluble portion using a gentle method of filtration (syringe equipped with a filter at 0.45 μm) then submitted to SEC analysis. Varying injection volumes are tested to obtain an optimal signal to noise ratio of at least five to one and to prevent and avoid overloading the SEC columns. Similarly, sample dilution could be slightly adjusted to obtain this optimal signal to noise ratio. The HPLC-SEC system used a dual set of mixed bed SEC columns suitable for the collection of a wide range of hyaluronic acid molecular weights from 500 Da to 20 MDa and for optimal resolution of the peaks on the chromatograms. In order to have a proper absolute molecular weight analysis of the sample using MALS detector, a do/dc value of 0.165 mL/g was determined based on in-house samples analysed using the same conditions as the samples. Further, in order to gauge the HPLC-SEC system equipped with the MALS and RI detectors, molecular weight polymer or protein standards are used and should be within 5% of the manufacturer identified molecular weight to ensure proper instrument performances.

Chromatograms obtained through SEC are analysed in order to quantify the molecular weight, distribution and proportion of soluble hyaluronic acid of each sample.

The mass-average molecular weight of a sample is a direct output of the HPLC-SEC software. The percentage of soluble hyaluronic acid fractions (% sHA) is also a direct output of the HPLC-SEC software after entering the total hyaluronic acid concentration of the analysed sample. Said percentage may differ from one composition to another according its manufacturing technique and the dispersity (p/d) of the sample.

The % sHA for multiple molecular weight limits (<250 kDa, <100 kDa, <50 kDa, <30 kDa and <20 kDa) are also a direct output of the HPLC-software. However, this does not take into account the amount of hyaluronic acid effectively released from the gel. As a result, a normalized % sHA for the multiple molecular weight limits has been calculated to normalize the values provided by the software by considering the amount of hyaluronic acid effectively released from the analysed sample. It is then possible to directly compare the results.

The “normalized % sHA for multiple Mw limits” indicates the proportion of soluble hyaluronic acid of molecular weight lower than 50 kDa, lower than 30 kDa and lower than with respect to the total weight of hyaluronic acid within the composition.

Modification Degree

The degree of modification is determined by Nuclear Magnetic Resonance spectroscopy (¹H NMR).

The gels were precipitated in isopropanol and dried for 6 hours under vacuum. The dried HA residues were dissolved at 10 mg/mL in D20. Hyaluronidase of 50 mL (Type VI-S from bovine testes, 3 kU/mL in D20) was added to degrade the gels for 18 hours at 37° C. The analysis was conducted on a 400 MHz Bruker Avance spectrometer.

The degree of modification was determined by the molar ratio of the crosslinking agent signals overs the hyaluronic acid disaccharide units signal (hyaluronic acid being crosslinked or non-crosslinked).

Analysis of Mechanical Performances of the Gels

Oscillatory Rheology Measurement

Mechanical Properties

A DHR2 rheometer (TA Instruments, software TRIOS a rough Peltier plate and rough parallel plate geometry (50 and 25-mm diameter, 250-mm rough, stainless steel, PMP Mecanique de Precision, France) was used for dynamic oscillatory rheological measurements.

0.50 g of each sample to be analysed was extruded through the syringe with the needle provided by the manufacturer on the rheometer plate. An oscillatory stress sweep test was performed at 25° C., over a stress range of 1 to 1,500 Pa at the oscillation frequency of 1 Hz, covering stresses within and beyond the Linear Visco-Elastic Region (LVER).

A preconditioning step was performed to equilibrate the gel at the working temperature for 70 seconds and at a working gap of 500 mm between the geometry and the rheometer plate. The values of the elastic modulus G′, the viscous modulus G″, the viscoelastic parameter δ, and the complex viscosity η* were measured at a stress of 5 Pa.

LVER and G′ Integration Score

The range of stresses from 1 Pa up to the stress value (in Pa) for which a decrease of 10% of initial G′ is considered as the LVER of a gel. It was verified that this decrease was not an artefact, that is, the G′ continued to drop when stress further increased.

The G′ integration score, Strength score, is calculated by integrating the area under the curve of G′ over LVER.

Creep Measurement

The Stretch test was performed using a creep measurement that consisted in applying a constant shear stress on the gels at 25° C. and measuring the resulting deformation over time. A preconditioning step was performed to equilibrate the gel at a working temperature of 25° C. during 70 seconds and at a working gap of 0.5 mm between the parallel flat plate geometry. After equilibrium, the Stretch test was performed at a stress of 5 Pa at 25° C. with the same gap for 900 seconds. The deformation curve was obtained, and the Stretch score was calculated from the slope of the steady-state viscous creep deformation part of the strain curve.

Cohesivity Scores

The assay of shear cohesivity in PBS (Phosphate Buffer Saline) is performed at 22±1° C. First, 1 g of each gel to be analysed is transferred into a 1 mL plastic syringes (Schott TopPac, Schott). A 15 g/L stock solution of methylene blue of 2 mL is placed in other syringes.

The syringe with gel sample and the other one with methylene blue are connected together and a series of back-and-forth extrusion cycles were performed to homogeneously stain gels without incorporating air bubbles. Stained gels were stored vertically at 6° C. overnight. For cohesivity tests, gels were extruded from the needle-less syringe in a 500 mL glass beaker containing 300 mL PBS, pH=7.3 (Braun Medical AG, Crissier, Switzerland).

Immediately after the gel extrusion, gel coils were gently and constantly stirred for 30 seconds, by pouring an extra 200 mL PBS in the beaker at an average flow rate of 6.7 mL/s. Video recordings of the gel under shear started at the same time as the gel stirring. Gel cohesion/dispersion was then visually assessed by 5 scientists, blinded to the product being assessed, according to the proposed Gavard-Sundaram cohesivity scale. The results are reported as the mean score±SDs.

This test was developed to probe the projection capacity and firmness of a gel. Gels intended for deeper indications such as volumizers are expected to display high mechanical resistance to resist tissue stress.

Compression Test

A DHR2 rheometer (TA Instruments, software TRIOS) equipped with a parallel flat plate geometry (40-mm diameter, anodized aluminum, TA Instruments, France) was used for mechanical compression assessments.

2 g of a gel to be analysed is deposited on enter of the Peltier plate at 25° C. The initial gap was set to 2.60 mm, and the gel was left to recover for 60 seconds. The gel was then compressed at a constant speed of 100 mm/s to 70% of the initial gap to limit gel expulsion from the geometries. The gel's mechanical resistance to compression is measured at the end of the compression course.

### Example 1: Molecular Weights Analysis of Soluble Hyaluronic Acid of Gels

Table 2 lists batch numbers of investigated gels and Table 3 indicates soluble hyaluronic acid characterization using HPLC-SEC-MALS.

TABLE 2

| Product ID | Batch number |
|---|---|
| Teosyal RHA 1 | TPRL-200621B |
| (RHA1) | |
| Teosyal RHA 2 | TP30L-200211A |
| (RHA2) | |
| Teosyal RHA 3 | TP27L-200311A |
| (RHA3) | |
| Teosyal RHA 4 | TPUL-200321B |
| (RHA4) | |
| Juvéderm Volite | V12LA90739 |
| (VYC-12L) | |
| Juvéderm Volbella | V15LA90261 |
| (VYC-15L) | |
| Juvéderm Volift | V17LA90320 |
| (VYC-17.5L) | |
| Juvéderm Voluma XC | VB20A90613 |
| (VYC-20L) | |
| Juvéderm Ultra XC | H24LA90517 |
| Juvéderm Ultra Plus XC | H30LA90276 |
| Restylane Skinboosters Vital | 17633-1 |
| (RES_{SV}) | |
| Restylane Lidocaine | 17604-1 |
| (RES) | |
| Restylane Lyft | 17460-1 |
| (RES_{LYFT}) | |
| Restylane Refyne | 17523 |
| (RES_{REF}) | |
| Restylane Defyne | 17360 |
| (RES_{DEF}) | |

TABLE 3

| | | p/d | | Normalized % sHA | | | | |
|---|---|---|---|---|---|---|---|---|
| | Mw | (Mw/ | % | 20 | 30 | 50 | 100 | 250 |
| Product ID | (kDa) | Mn) | sHA | kDa | kDa | kDa | kDa | kDa |
| Teosyal | 665 | 1.92 | 36.20 | 0 | 0 | 0.07 | 1.80 | 6.80 |
| RHA 1 | | | | | | | | |
| (RHA1) | | | | | | | | |
| Teosyal | 596 | 3.02 | 24.95 | 0 | 0 | 0.90 | 4.38 | 9.19 |
| RHA 2 | | | | | | | | |
| (RHA2) | | | | | | | | |
| Teosyal | 496 | 3.56 | 16.93 | 0 | 0 | 1.39 | 4.78 | 8.72 |
| RHA 3 | | | | | | | | |
| (RHA3) | | | | | | | | |
| Teosyal | 599 | 4.20 | 18.73 | 0 | 0.02 | 1.85 | 5.19 | 8.25 |
| RHA 4 | | | | | | | | |
| (RHA4) | | | | | | | | |
| Juvéderm | 161 | 2.93 | 16.00 | 0 | 2.16 | 6.22 | 11.43 | 14.50 |
| Volite | | | | | | | | |
| (VYC-12L) | | | | | | | | |
| Juvéderm | 165 | 3.08 | 24.12 | 1.64 | 3.96 | 8.08 | 15.29 | 20.78 |
| Volbella | | | | | | | | |
| (VYC-15L) | | | | | | | | |
| Juvéderm | 83 | 2.55 | 26.50 | 4.37 | 7.02 | 11.63 | 18.47 | 24.95 |
| Volift | | | | | | | | |
| (VYC- | | | | | | | | |
| 17.5L) | | | | | | | | |
| Juvéderm | 88 | 1.55 | 26.70 | 0 | 2.40 | 9.74 | 19.72 | 25.62 |
| Voluma XC | | | | | | | | |
| (VYC-20L) | | | | | | | | |
| Juvéderm | 298 | 2.56 | 29.50 | 0 | 0 | 2.93 | 9.73 | 19.20 |
| Ultra XC | | | | | | | | |
| Juvéderm | 295 | 3.10 | 69.41 | 0 | 0.28 | 14.47 | 26.16 | 48.67 |
| Ultra Plus | | | | | | | | |
| XC | | | | | | | | |
| Restylane | 189 | 1.69 | 27.77 | 0 | 0 | 2.37 | 9.09 | 20.90 |
| Skinboosters | | | | | | | | |
| Vital | | | | | | | | |
| (RESₛᵥ) | | | | | | | | |
| Restylane | 200 | 1.60 | 24.08 | 0 | 0 | 0.90 | 6.95 | 18.14 |
| Lidocaine | | | | | | | | |
| (RES) | | | | | | | | |
| Restylane | 188 | 2.07 | 27.47 | 0.74 | 1.46 | 4.00 | 8.69 | 21.72 |
| Lyft | | | | | | | | |
| (RES_{LYFT}) | | | | | | | | |
| Restylane | 271 | 1.83 | 38.20 | 0 | 0 | 0 | 9.43 | 26.24 |
| Refyne | | | | | | | | |
| (RES_{REF}) | | | | | | | | |
| Restylane | 93 | 1.86 | 32.81 | 2.43 | 4.95 | 10.61 | 21.34 | 31.55 |
| Defyne | | | | | | | | |
| (RES_{DEF}) | | | | | | | | |

We can see in Table 3 and FIG. 1A that TEOSYAL RHA gels released soluble hyaluronic acids, on average, about twice longer than Restylane Lidocaine (RES) and even three times longer than Juvéderm gels prepared with the technology Vycross.

We can see in Table 3 and FIG. 1B that the percentage by weight of released soluble hyaluronic acid with respect to the total weight of hyaluronic acid within the composition ranged between 16 and 38% independently of the gel indications or manufacturing technology, with the exception of Juvéderm Ultra Plus with a ratio of 70%. No clear trend could be extracted from this parameter taken alone. Nevertheless, this value is of paramount importance to normalize the % sHA for each molecular weight limits.

Table 3 and FIG. 1C show the quantity of soluble hyaluronic acid fragments released from the studied gels for the molecular weight limits of <250 kDa, <100 kDa, and <30 kDa.

The percentage by weight of soluble hyaluronic acid with respect to the total weight of hyaluronic acid within the composition:
with a molecular weight lower than 250 kDa ranged between 6.8% for RHA 1 to 31.5% for RES_{DEF},
with a molecular weight lower than 100 kDa ranging between 1.8% for RHA 1 to 11.6% for RES_{DEF}, and
with a molecular weight lower than 30 kDa ranged between 0% for RHA product line, RES_{SV}, RES and RES_{REF} to 7.0% for VYC-17.5L.

TEOSYAL RHA products exhibited the lowest proportion of soluble hyaluronic acid having a molecular weight lower than 250 kDa, with:
less than 9.2% by weight of the total weight of hyaluronic acid within the composition being soluble hyaluronic acid with a molecular weight lower than 250 kDa,
less than 5.2% by weight of the total weight of hyaluronic acid within the composition being soluble hyaluronic acid with a molecular weight lower than 100 kDa, and
no soluble hyaluronic acid with a molecular weight lower than 30 kDa.

VYC and RES products presented similar contents of soluble hyaluronic acid having a molecular weight lower than 250 kDa with:
14.5 to 31.5% by weight of the total weight of hyaluronic acid within the composition being soluble hyaluronic acid with a molecular weight lower than 250 kDa,
7.0 to 21.3% by weight of the total weight of hyaluronic acid within the composition being soluble hyaluronic acid with a molecular weight lower than 100 kDa, and,
0 to 7% by weight of the total weight of hyaluronic acid within the composition being soluble hyaluronic acid with a molecular weight lower than 30 kDa.

The analysis of the results presented in Table 3 and FIG. 1(A, B, C) allows the following conclusions:
No clear trend could be obtained using The ratio of the total amount of soluble hyaluronic acid to the total amount of hyaluronic acid within each studied compositions alone.
Teosyal RHA 1, Teosyal RHA 2, Teosyal RHA 3, Teosyal RHA 4, Juvéderm Ultra XC, Restylane Skinboosters Vital, Restylane Lidocaine, Restylane Lyft, and Restylane Refyne present low proportions of soluble hyaluronic acid having a molecular weight lower than kDa (proportion lower than 5% by weight with respect to the total weight of hyaluronic acid within the composition), the same gels present a low proportion of soluble hyaluronic acid having a molecular weight lower than 30 kDa (proportion lower than 2% by weight with respect to the total weight of hyaluronic acid within the composition) and a low proportion of soluble hyaluronic acid having a molecular weight lower than 20 kDa (proportion lower than 1% by weight with respect to the total weight of hyaluronic acid within the composition). Further, Teosyal RHA products range exhibits the highest mass-average molecular weights, with mass-average molecular weights higher than 300 kDa, suggesting a better safety profile in vivo of Teosyal RHA products in comparison with Juvéderm and Restylane products.
Only Restylane Lyft, Restylane Defyne, Juvéderm Volbella and Juvéderm Volift present soluble hyaluronic acid of molecular weight of less than 20 kDa, suggesting their particularly unfavorable safety profile in vivo.
Finally, Teosyal RHA products exhibit mass-average molecular weights of soluble hyaluronic acid higher than every other tested products.

### Example 2: Mechanical, Rheological, and Chemical Analysis of the Gels

The same library of gels is mechanically, rheologically, and chemically analysed. Table 4 lists batch numbers of investigated gels.

TABLE 4

| Product ID | Batch number |
|---|---|
| Teosyal RHA 1 | TPRL-171213A |
| (RHA 1) | |
| Teosyal RHA 2 | TP30L-171117A |
| (RHA 2) | |
| Teosyal RHA 3 | TP27L-172417A |
| (RHA 3) | |
| Teosyal RHA 4 | TPUL-172616B |
| (RHA 4) | |
| Juvéderm Volite | V12LA70463 |
| (VYC-12L) | |
| Juvéderm Volbella | V15LA70599 |
| (VYC-15L) | |
| Juvéderm Volift | V17LA70608 |
| (VYC-17.5L) | |
| Juvéderm Voluma XC | VB20A70598 |
| (VYC-20L) | |
| Juvéderm Ultra XC | H24LA90517 |
| Juvéderm Ultra Plus XC | H30LA90158 |
| Restylane Skinboosters Vital | 15598-1 |
| (RES_{SV}) | |
| Restylane Lidocaine | 14560-2 |
| (RES) | |
| Restylane Lyft | 15585-1 |
| (RES_{LYFT}) | |
| Restylane Refyne | 17522 |
| (RES_{REF}) | |
| Restylane Defyne | 17525 |
| (RES_{DEF}) | |

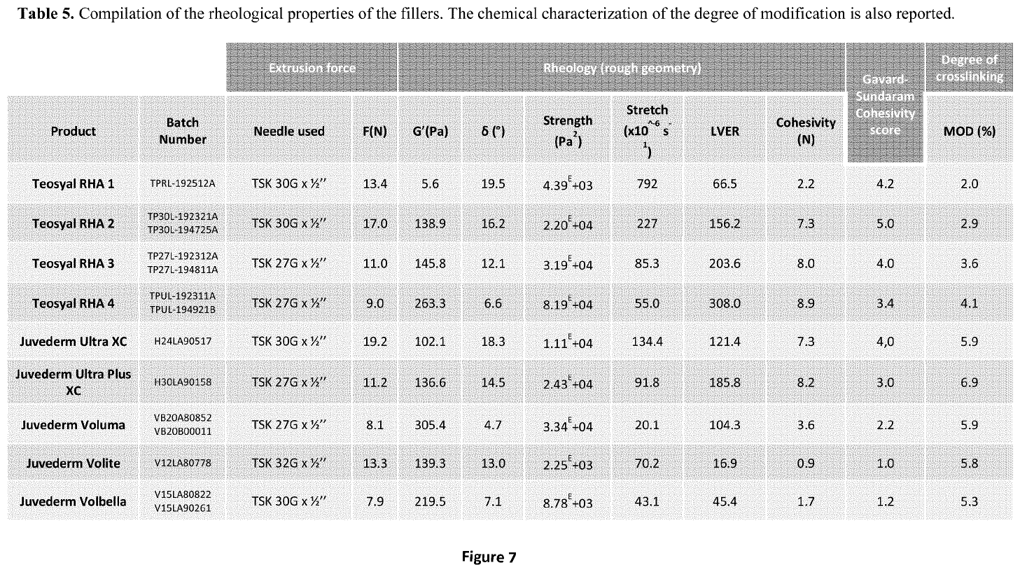

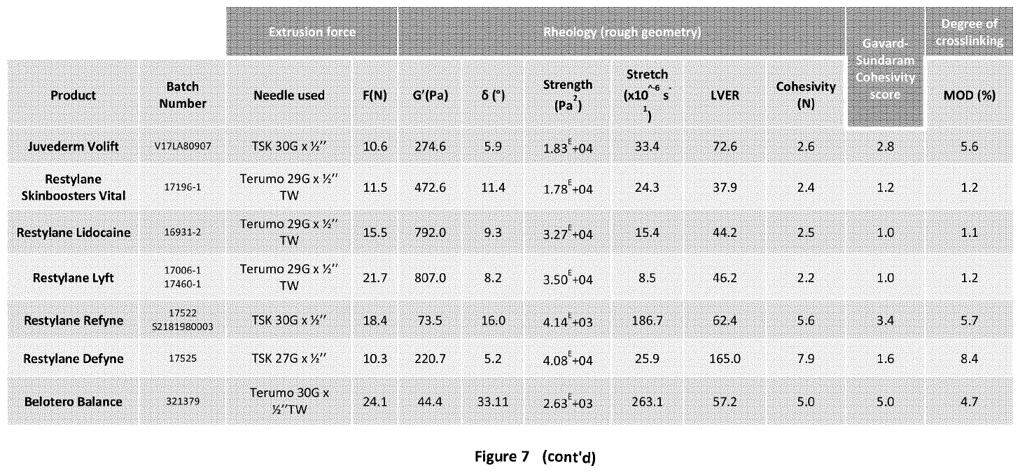

The results are displayed in Table 5 (FIG. 7 showing rheological properties of the gels and their degree of modification) and in FIGS. 1D to 6.

FIG. 1D shows the MoD of the studied gels. NASHA products present the lowest MoD values (between 1.1%-1.2% for RES_{SV} RES and RES_{LYFT}). RHA products exhibit MoD ranging between 2.0% to 4.1%. The MoD of Vycross products range between 5.3% and 5.9%, similarly to RES_{REF}. RES_{DEF} presents the highest MoD of the investigated products at 8.4%.

It can be seen in FIGS. 2B and 2C that, after mild shear stirring of a gel into an aqueous buffered solution, RHA products exhibit the highest cohesivity among all investigated gels. On the other hand, VYC-12L, RES and RES_{LYFT} present the lowest cohesivities.

Results presented in FIGS. 3B and 3C highlight mechanical resistance of the gels thanks to the compression test. Unlike RES_{LYFT}, the gels intended for volumizing (RHA 4, VYC-RES_{DEF}) exhibit the highest resistance to compression. RHA 4 presents the highest value of all the investigated gels.

FIGS. 4A and 4B show the common viscoelastic parameters: the elastic modulus G′, the phase angle δ and the complex viscosity η* of investigated gels. Regarding the most used parameter, G′, RHA, VYC, RES_{REF}, and RES_{DEF} present a similar range, whereas RES_{SV}, RES, and RES_{LYFT} present the highest G′ values. This trend did not follow the cohesivity and mechanical resistance macroscopic test, thus highlighting the gap between G′ measurement at nearly static conditions and the fact that gels are implanted in dynamic living regions which do not match the G′ measurement conditions.

FIG. 4C presents the width of the LVER. Here, RHA products line produces the larger LVER, especially RHA3 and RHA4 present the largest LVER, meaning that they will withstand their elastic modulus G′ over the largest ranges of stresses, thus being able to lift tissues even in dynamic zones.

FIG. 4D displays typical G′ curves plotted against stress for the volumizers VYC-20L, RES_{LYFT} and RHA 4. It shows that RES_{LYFT} exhibit the largest G′ value (about 800 Pa), but on the lowest LVER (about 50 Pa). It means that at a very low and almost imperceptible value of 50 Pa, this gel starts to lose its viscoelastic properties. On the contrary, VYC-20_{L} and RHA 4 present lower G′ values (between 260-300 Pa), but on larger LVER (about 100 Pa for VYC-20_{L} and 300 Pa for RHA 4). This means that RHA 4 will provide its viscoelastic properties on a stress range 6 times larger than RES_{LYFT} and 3 times larger than VYC-20L. Further, this curve helps to calculate the G′ integration score.

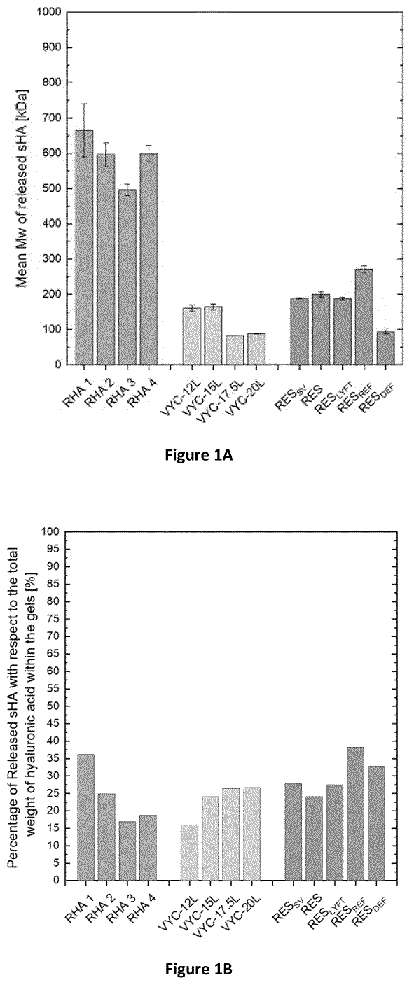

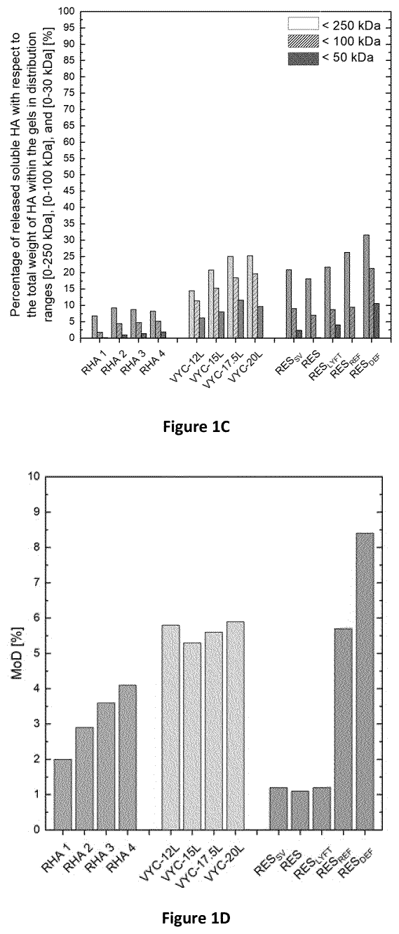

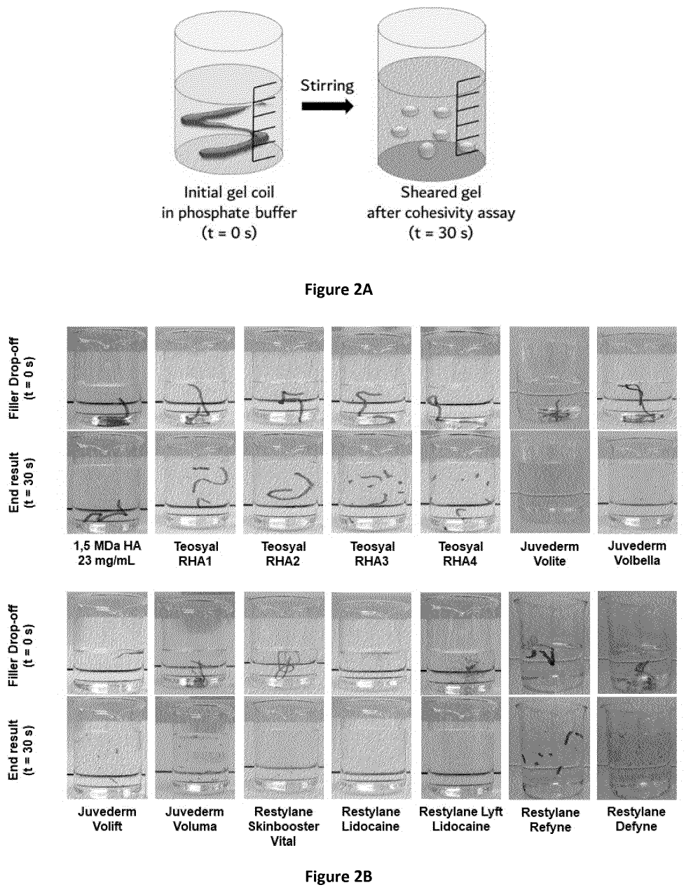

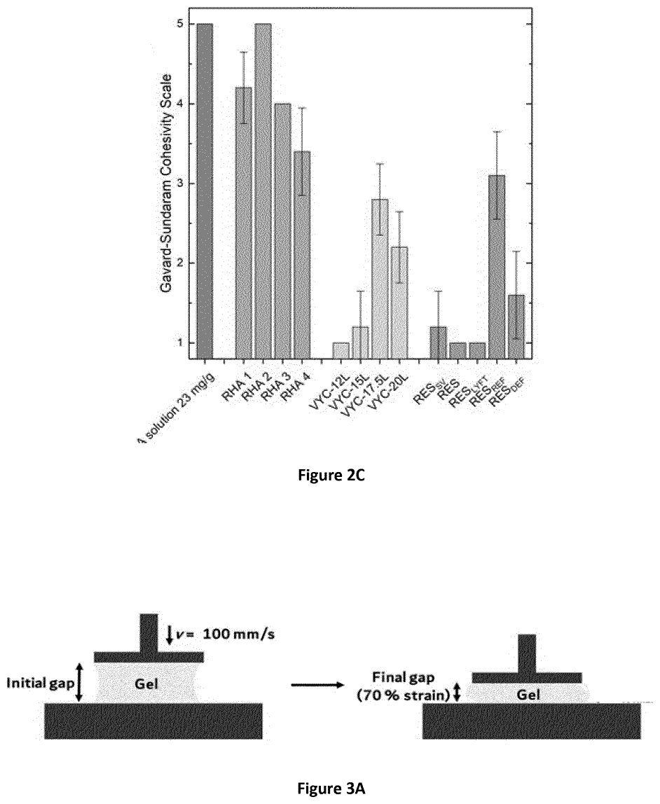

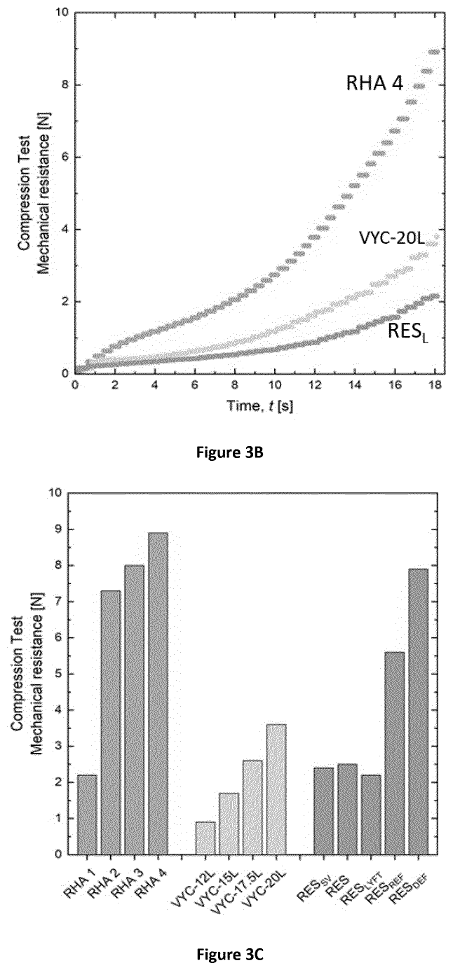

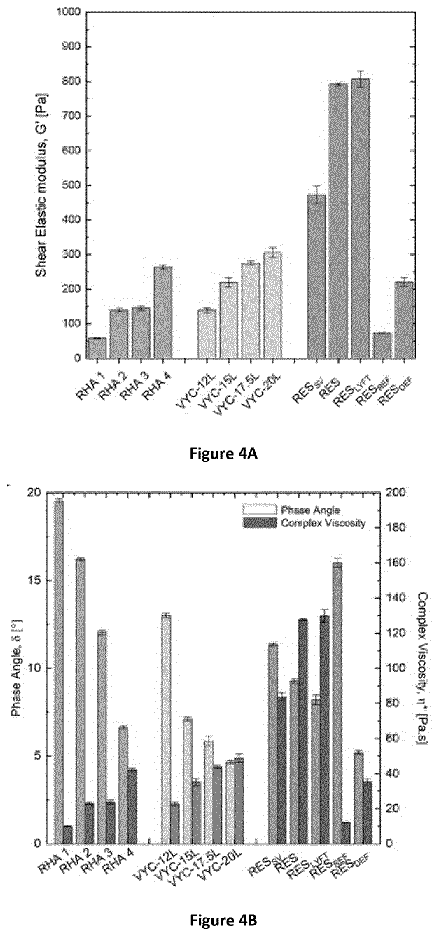

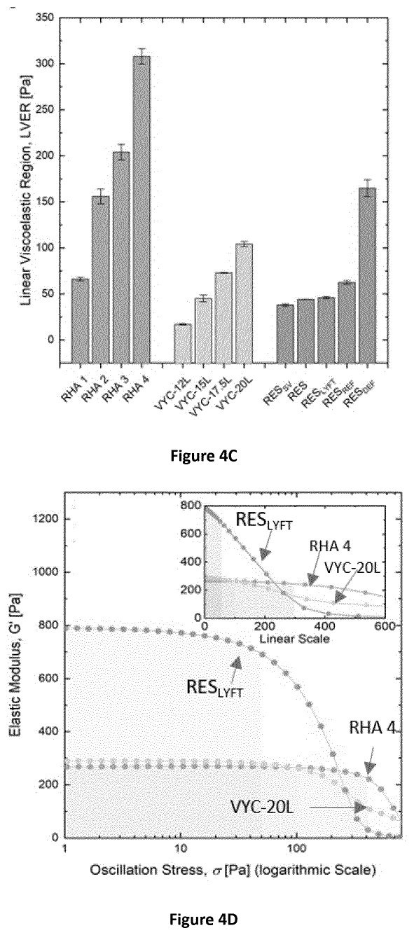

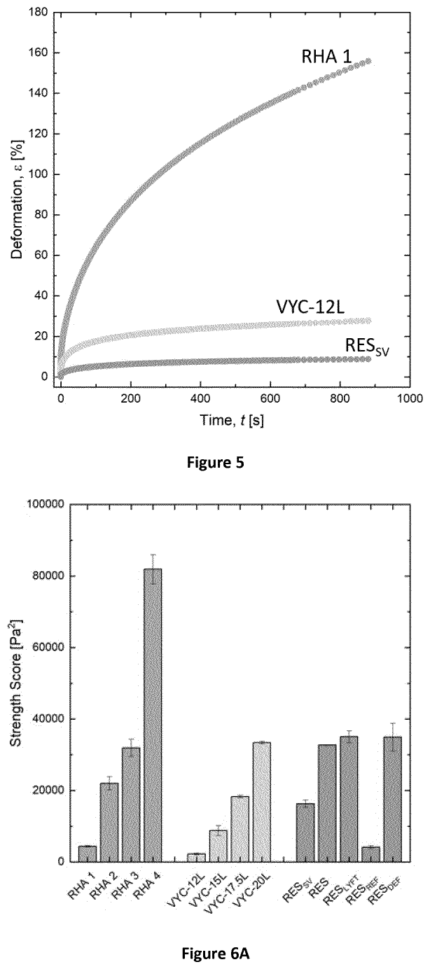

FIG. 5 displays the deformation curve resulting from the creep measurement of RHA 1, VYC-12L, and RES_{SV}, that are gels intended for superficial wrinkles filling needing to specifically adapt tissue motion to provide natural-looking results.

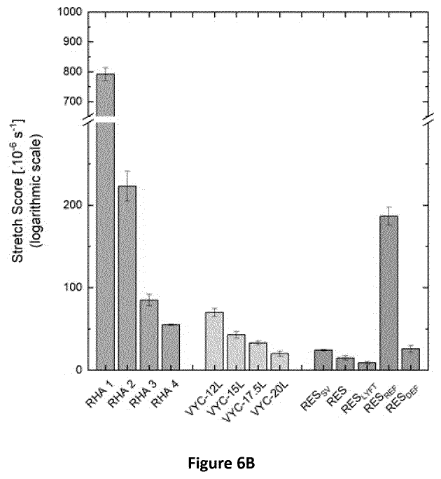

As it can be seen on FIG. 6A regarding the Strength score, RHA 4 presents the highest value (about 80,000 Pa²) followed by RHA 3, VYC-20L, RES, RES_{LYFT}, and RES_{DEF} with similar values (around 30,000-35,000 Pa²). Gels intended for more superficial indications present lower Strength scores, notably RHA 1, VYC-12L, and RES_{SV}.

Regarding the Stretch score, gels for more superficial indications show the largest capacity of malleability among the investigated gels with RHA 1 presenting the highest score (about 800.10⁻⁶ s⁻¹) followed by RHA 2 (about 220.10⁻⁶ s⁻¹) then RES_{REF} (about 190.10⁻⁶ s⁻¹) (FIG. 6B).

### Example 3: Selection of the Most Suitable Gels Depending on its Indications

The selection comprises the selection of gels which display low amounts of normalized % sHA for the molecular weight limits:
less than 5% by weight of soluble hyaluronic acid having a molecular weight lower than 50 kDa with respect to the total weight of hyaluronic acid within the composition, and/or,
less than 2% by weight of soluble hyaluronic acid having a molecular weight lower than 30 kDa with respect to the total weight of hyaluronic acid within the composition, and/or,
less than 1% by weight of soluble hyaluronic acid having a molecular weight lower than 20 kDa with respect to the total weight of hyaluronic acid within the composition.

According to hereabove Table 3, Teosyal RHA 1, Teosyal RHA 2, Teosyal RHA 3, Teosyal RHA 4, Juvéderm Ultra XC, Restylane Skinboosters Vital, Restylane Lidocaine, Restylane Lyft, and Restylane Refyne can be selected.

The next step is to select gels having soluble hyaluronic acid with the highest mass-average molecular weight (higher than or close to 300 kDa). According to Table 3, the remaining gels are thus Teosyal RHA 1, Teosyal RHA 2, Teosyal RHA 3, Teosyal RHA 4, Juvéderm Ultra XC, and Restylane Refyne.

Finally, depending on their intended uses, gels are selected according to their Stretch and/or Strength scores.

For Superficial Indications:

Malleability and adaptability of the gel is of paramount importance to offer natural-looking results. Thus, gels with Stretch scores higher than ≥100.10⁻⁶ s⁻¹, more preferably ≥150.10⁻⁶ s⁻¹, still more preferably ≥200.10⁻⁶ s⁻¹ are selected. Therefore, Teosyal RHA 1 and Teosyal RHA 2 are here the only two remaining gel candidates. A further distinction can be done based on the specific final indication for the gel. If the gel is intended for the more superficial layers of the skin but still to have some lifting capacities, Teosyal RHA 2 having a higher Strength score than Teosyal RHA 1 will be used. Otherwise, if the gel is only intended for the more superficial layers of the skin, Teosyal RHA 1 will be selected as it has the highest Stretch score.

For Volumizing Indications:

Gels having the highest G′ integration scores (e.g. higher than or equal to 30.000 Pa²) are selected to withstand the stronger stress met in deeper tissues. Based on this, Teosyal RHA 4 will be selected.

1. A method of selecting composition(s) comprising crosslinked hyaluronic acid and soluble hyaluronic acid from a set of compositions comprising crosslinked hyaluronic acid and soluble hyaluronic acid, comprising the steps of:
a) extracting the soluble hyaluronic acid of each composition of the set of compositions by diluting each composition within a solvent usable as mobile phase for Size Exclusion Chromatography (SEC) in order to obtain a diluted composition and filtering each such diluted composition to obtain a filtrate;
b) injecting each filtrate obtained through step a) in SEC column(s) and eluting it through the column(s) to obtain chromatograms;
c) analyzing the chromatograms obtained through step b) in order to identify and quantify the soluble hyaluronic acid molecular weights, and notably to determine the amount of the soluble hyaluronic acid having a molecular weight lower than 50 kDa, the amount of the soluble hyaluronic acid having a molecular weight lower than 30 kDa, the amount of the soluble hyaluronic acid having a molecular weight lower than 20 kDa, and optionally the weight average molecular weight of the soluble hyaluronic acid, and,
d) thanks to step c) analysis, selecting composition(s) having:
the lower amount(s) of the soluble hyaluronic acid having a molecular weight lower than 50 kDa in percentage by weight with respect to the total weight of the hyaluronic acid within the composition, or an amount inferior or equal to 5%, preferably inferior or equal to 4%, still preferably inferior or equal to 3%, better still preferably inferior or equal to 2%, by weight with respect to the total weight of the hyaluronic acid within the composition; and/or
the lower amount(s) of the soluble hyaluronic acid having a molecular weight lower than 30 kDa in percentage by weight with respect to the total weight of the hyaluronic acid within the composition, or an amount inferior or equal to 2%, preferably inferior or equal to 1%, by weight with respect to the total weight of the hyaluronic acid within the composition; and/or
the lower amount(s) of the soluble hyaluronic acid having a molecular weight lower than 20 kDa in percentage by weight with respect to the total weight of the hyaluronic acid within the composition, or an amount inferior or equal to 1% with respect to the total weight of the hyaluronic acid within the composition.

2. The method according to claim 1, wherein step d) comprises selecting composition(s) with the soluble hyaluronic acid having the highest mass-average molecular weight(s), or having a mass-average molecular weight higher than 300 kDa, wherein the mass-average molecular weight of the soluble hyaluronic acid is determined in step c).

3. The method according to claim 1 or 2, comprising, in step d), selecting composition(s) comprising at least 50% by weight of insoluble hyaluronic acid with respect to the total weight of the hyaluronic acid within the composition.

4. The method according to any one of claims 1 to 3, comprising an additional step e) of evaluating the mechanical performance of each composition of the set of compositions and an additional step f) of selecting composition(s) according to its (their) mechanical performances.

5. The method according to claim 4, wherein step e) comprises submitting composition(s) to:
oscillatory rheology to determine their elastic modulus (G′) and to deliver a G′ integration score, and/or to
a creep measurement to determine the slope of their deformation curve.

6. The method according to claim 4 or 5, wherein in step f) the composition(s) is(are) selected for having:
the highest G′ integration score; or
a G′ integration score higher than or equal to 30.000 Pa e.

7. The method according to any one of claims 4 to 6 wherein in step f) the composition(s) is(are) selected for having
the highest slope of the deformation curve; or
a slope of the deformation curve ≥100.10⁻⁶ s⁻¹, more preferably ≥150.10⁻⁶ s⁻¹, still more preferably ≥200.10⁻⁶ s⁻¹.

8. The method according to any one of the preceding claims, wherein the set of compositions consists in a set of gels.
